(19) United States
(12) Patent Application Publication
(10) Pub. No.: US 2026/0167741 A1
(43) Pub. Date: Jun. 18, 2026

(54) HUMAN ANTIBODY FC DOMAIN VARIANT AND USE THEREOF

(71) Applicant: CROSSPOINT THERAPEUTICS INC., Gyeonggi-do (KR)

(72) Inventors: SANG TAEK JUNG, Seoul (KR); SU YEON KIM, Seoul (KR); MI GYEONG JO, Seoul (KR)

(21) Appl. No.: 19/122,362

(22) PCT Filed: Oct. 18, 2023
(86) PCT No.: PCT/KR2023/016105

§ 371 Date: Apr. 18, 2025

(30) Foreign Application Priority Data
Oct. 18, 2022 (KR) 10-2022-0133722

(51) Int. Cl. C07K 16/32; A61P 35/00

(52) U.S. Cl. CPC C07K 16/32; A61P 35/00; C07K 2317/52; C07K 2317/92

(57) ABSTRACT

A human antibody Fc domain variant has proline (P) substituted for the 235th amino acid of the wild-type human antibody Fc domain and thus lacks or has substantially no binding ability to human FcγRs and C1q, with the consequent deficiency of antibody-dependent cellular cytotoxicity (ADCC), antibody-dependent cell-mediated phagocytosis (ADCP), and complement-dependent cytotoxicity (CDC).

### REFERENCE TO AN ELECTRONIC SEQUENCE LISTING

A sequence listing electronically submitted with the present application on Apr. 18, 2025 as a XML file named 20250418_547725LC05_TU_SEQ.XML, created on Apr. 17, 2025 and having a size of 26,401 bytes, is incorporated herein by reference in its entirety.

### BACKGROUND

### 1. Technical Field

The present invention relates to a human antibody Fc domain variant and a use thereof.

### 2. Background Art

Since protein therapeutics show very high specificity for disease targets and low side effects and toxicity, they are rapidly replacing non-specific small molecule compound therapeutics and are widely used in clinical practice. Among protein therapeutics currently used clinically, antibody therapeutics are Fc-fusion protein therapeutics that are fused with the Fc region of an antibody are mainly used.

Therapeutic antibodies are considered one of the most effective cancer treatment methods because they show very high specificity for a target compared to existing small molecule drugs, have low biotoxicity and fewer side effects, and have an excellent blood half-life of about 3 weeks. In fact, large pharmaceutical companies and research institutes around the world are accelerating the research and development of therapeutic antibodies that specifically bind to cancer cells, including cancer-causing factors, and effectively remove the same.

Antibodies provide a link between the humoral and cellular immune systems; while the Fab region of an antibody recognizes an antigen. On the other hand, the Fc domain portion is bound to a receptor (Fc receptor or FcR) to antibodies (immunoglobulins) on cells that are differentially expressed by all immunocompetent cells, and has different mechanisms depending on the type of FcγR expressed on the surface of the immune cell to which it binds. The Fc receptor binding site on the antibody Fc region binds to the Fc receptor (FcR) on the cell, such that, when the antibody binds to the Fc receptor on the cell surface through the Fc region, it triggers several important and diverse biological reactions, including phagocytosis and destruction of antibody-coated particles, removal of immune complexes, lysis of antibody-coated target cells (antibody-dependent cellular cytotoxicity, or ADCC) by killer cells, release of inflammatory mediators, placental migration and control of immunoglobulin production.

The Fc domain plays a critical role in the collection of immune cells, the antibody-dependent cellular cytotoxicity (ADCC) and antibody-dependent cellular phagocytosis (ADCP). In particular, the ADCC and ADCP functions, which are the effector functions of antibodies, rely on interaction with Fc receptor present on the surface of many cells. Human Fc receptors are classified into five types, wherein the type of collected immune cells is determined depending on which Fc receptor the antibody binds to. For example, the Fc domain of an antibody binds to FcγRIIIa to induce ADCC, binds to FcγRI or FcγRIIa to induce ADCP, and binds to blood complement C1q to activate a complement dependent cytotoxicity (CDC) action mechanism, thereby being toxic to the target antigen bound to Fab region. Therefore, it is responsible for the main therapeutic effects of therapeutic antibodies.

The mechanism of action by this antibody Fc region induces a death mechanism of defective cells (e.g., cancer, infected cells), and is an important action mechanism of therapeutic antibodies. However, in the case where a target antigen of a therapeutic antibody such as immune checkpoint inhibitor antibody that binds to immune cells, immune cell-derived double antibody (bispecific immune cell engaging bispecific antibody), etc. is expressed on immune cells rather than defective cells (cancer, infected cells), if the Fc action mechanism remains, normal immune cells bound with the antibody, which are beneficial for immune action, may be destroyed or activate host immune defenses, which may lead to safety issues and undesired side effects.

For example, some therapeutic antibodies, such as immune checkpoint inhibitors that bind to immune cells, bispecific immune cell engaging bispecific antibody (abbrev. to bispecific antibody), etc., have a problem of side effects that destroy immune cells by triggering an immune action mechanism on targeted immune cells. Further, the immune checkpoint inhibitor (e.g., Keytruda, Opdivo, Libtayo, etc.) targeting an immune checkpoint protein expressed on the surface of immune cell having T-cell entails a disadvantage of deteriorating original effects of the antibody due to side effects in that, if Fc-mediated action mechanism remains, the immune cells themselves, which should be used to kill cancer cells, are destroyed.

Further, the bispecific antibody as an antibody therapeutic agent, which binds to an antigen on the surface of a cancer cell on one side while being bound to an immune cell on the other side, and which may act to guide the immune cell to the cancer cell thus to more efficiently remove the cancer cell, causes side effects because the immune cell is destroyed and the cancer cell could not be effectively removed if Fc-mediated immune action mechanism is present in the corresponding antibody. Further, agonist antibodies that bind to target cells to induce cell activation, or antagonist antibodies that block an interaction between the target antigen and the ligand are toxic to target cells and antigens due to the Fc-mediated immune action mechanism, hence causing a problem of deteriorating the original effect of the antibody. In addition, when developing an Fc-fusion protein in which the Fc region is fused to increase the half-life of an active substance such as a protein or compound according to purposes for therapeutic, diagnostic or research, there is a problem of appearing toxicity due to the Fc-mediated immune action mechanism.

Therefore, in order to solve the problem of causing immune cell death side effects/off-target toxicity to normal cells while having excellent efficacy of the antibody due to target antigen-specific binding, removal of the Fc action mechanism is essential. To this end, when developing antibodies, IgG2 antibodies, which have the lowest binding ability to FcγR among the human IgG subclasses and thus have a very low immune action mechanism, are considered. However, IgG2 antibodies have several allotypes due to disulfide bond exchange in a hinge region. Further, there are physical property problems such as aggregation due to the decreased stability. Therefore, IgG4 antibodies with next lower binding ability are being considered and are currently being used in clinical development. However, the IgG4 antibodies also have a disadvantage of causing Fab-arm exchange of IgG antibody molecule due to lack of stability of antibody hinge region. Furthermore, these antibodies still have significant binding ability to FcγRs, and in particular, have quite strong binding ability to FcγRI (equilibrium dissociation constant of several nM), hence involving a problem with activation of various Fc action mechanisms. Accordingly, in order to prevent target cells from being destroyed by the immune action mechanism of the antibody, Fc with removed the binding ability to FcγRs is needed.

### SUMMARY

An object of the present invention is to provide a novel human antibody Fc domain variant.

Another object of the present invention is to provide a novel antibody with reduced effector function or a fragment thereof having immunological activity.

In addition, another object of the present invention is to provide a novel antibody-drug conjugate.

Further, another object of the present invention is to provide a pharmaceutical composition for treating or preventing cancer.

Furthermore, another object of the present invention is to provide a method for treating cancer.
1. A human antibody Fc domain variant in which amino acid 235 of the human antibody Fc domain is replaced with proline (P).
2. The human antibody Fc domain variant according to the above 1, wherein the human antibody Fc domain including an amino acid sequence of SEQ ID NO: 15.
3. The human antibody Fc domain variant according to the above 1, wherein amino acid 234 of the human antibody Fc domain is further substituted with alanine (A).
4. The human antibody Fc domain variant according to the above 1, wherein amino acid 265 of the human antibody Fc domain is further substituted with leucine (L), methionine (M) or asparagine (N).
5. The human antibody Fc domain variant according to the above 1, wherein amino acid 329 of the human antibody Fc domain is further substituted with valine (V), leucine (L), or glycine (G).
6. The human antibody Fc domain variant according to the above 1, wherein amino acid 268 of the human antibody Fc domain is further substituted with glutamine (Q).
7. The human antibody Fc domain variant according to the above 1, wherein amino acid 296 of the human antibody Fc domain is further substituted with phenylalanine (F).
8. The human antibody Fc domain variant according to the above 1, wherein amino acid 300 of the human antibody Fc domain is further substituted with phenylalanine (F).
9. The human antibody Fc domain variant according to the above 1, wherein amino acid 333 of the human antibody Fc domain is further substituted with valine (V).
10. The human antibody Fc domain variant according to the above 1, wherein the human antibody is IgG1.
11. An antibody including the human antibody Fc domain variant according to any one of the above 1 to 10, or a fragment thereof having immunological activity.
12. An antibody-drug conjugate in which the antibody according to the above 11 or a fragment thereof having immunological activity is conjugated to one or more drugs.
13. The antibody-drug conjugate according to the above 12, wherein the drug is any one selected from the group consisting of: chimeric antigen receptor (CAR) cell therapeutics, oncolytic drug, immunotherapy agent, cytotoxic agent, angiogenesis inhibitor, kinase inhibitor, costimulatory molecule blocker, adhesion molecule blocker, anti-cytokine agent, nucleic acid therapeutics, anti-CTLA-4 agent, anti-PD-1 agent, anti-PD-L1 agent, anti-PD-L2 agent, TNF-α cross-linking agent, TRAIL cross-linking agent, anti-CD27 agent, anti-CD30 agent, anti-CD40 agent, anti-4-1BB agent, anti-GITR agent, anti-OX40 agent, anti-TRAILR1 agent, anti-TRAILR2 agent, tagretin, interferon-alpha, clobetasol, peg interferon, prednisone, romidepsin, bexarotene, methotrexate, triamcinolone cream, anti-chemokine, vorinostat, gabapentin, cyclosporine, rapamycin, FK506, detectable marker or reporter, TNF antagonist, antirheumatic agent, muscle relaxants, narcotics, non-steroidal anti-inflammatory drugs (NSAIDs), analgesics, anesthetics, sedatives, local anesthetics, neuromuscular blocker, antibacterial agent, psoriasis therapeutics, corticosteroid, anabolic steroid, erythropoietin, immunizations, immunoglobulin, immunosuppressant, growth hormone, hormone replacement drug, radiopharmaceuticals, antidepressant, antipsychotics, stimulant, asthma drug, beta agonist, inhaled steroid, epinephrine or its analogues, cytokine, cytokine antagonist, PD-1 antagonist, adenosine A2AR antagonist, CD73 inhibitor, CTLA-4 inhibitor, TIM-3 inhibitor, LAG-3 inhibitor, anthracycline, antisense oligonucleotide (ASO), siRNA and aptamer.
14. A pharmaceutical composition for treatment or prevention of cancer, including: the human antibody Fc domain variant according to any one of the above 1 to 10; an antibody including the same or a fragment thereof having immunological activity; or the antibody-drug according to the above 12 or 13.
15. The pharmaceutical composition according to the above 14, wherein the cancer is selected from the group consisting of: brain tumor, melanoma, myeloma, non-small cell lung cancer, oral cancer, liver cancer, stomach cancer, colon cancer, breast cancer, lung cancer, bone cancer, pancreatic cancer, skin cancer, head or neck cancer, cervical cancer, ovarian cancer, colon cancer, small intestine cancer, rectal cancer, fallopian tube carcinoma, anal cancer, endometrial carcinoma, vaginal carcinoma, vulvar carcinoma, Hodgkin's disease, esophageal cancer, lymph node cancer, bladder cancer, gallbladder cancer, endocrine cancer, thyroid cancer, parathyroid cancer, adrenal cancer, soft tissue sarcoma, urethral cancer, penile cancer, prostate cancer, chronic or acute leukemia, lymphocytic lymphoma, renal or ureteral cancer, renal cell carcinoma, renal pelvic carcinoma, central nervous system tumor, primary central nervous system lymphoma, spinal cord tumor, brain stem glioma and pituitary adenoma.
16. A method for treatment of cancer, including: administering the human antibody Fc domain variant according to any one of the above 1 to 10, an antibody including the same or a fragment thereof having immunological activity; or the antibody-drug conjugate according to the above 12 or 13, to a subject.
17. The method according to the above 16, wherein the cancer is selected from the group consisting of: brain tumor, melanoma, myeloma, non-small cell lung cancer, oral cancer, liver cancer, stomach cancer, colon cancer, breast cancer, lung cancer, bone cancer, pancreatic cancer, skin cancer, head or neck cancer, cervical cancer, ovarian cancer, colon cancer, small intestine cancer, rectal cancer, fallopian tube carcinoma, anal cancer, endometrial carcinoma, vaginal carcinoma, vulvar carcinoma, Hodgkin's disease, esophageal cancer, lymph node cancer, bladder cancer, gallbladder cancer, endocrine cancer, thyroid cancer, parathyroid cancer, adrenal cancer, soft tissue sarcoma, urethral cancer, penile cancer, prostate cancer, chronic or acute leukemia, lymphocytic lymphoma, renal or ureteral cancer, renal cell carcinoma, renal pelvic carcinoma, central nervous system tumor, primary central nervous system lymphoma, spinal cord tumor, brain stem glioma and pituitary adenoma.
18. A composition for target detection, including a conjugate in which the antibody according to the above 11 or a fragment thereof having immunological activity and a functional substance are bound.
19. The composition for target detection according to the above 18, wherein the functional material is one or more selected from the group consisting of contrast agents, radioisotopes, inorganic particles, fluorescent markers, dyes, enzymes, polypeptides, nucleic acids, carbohydrates and lipids.
20. A method for diagnosing a disease, including administering the composition for target detection according to the above 18 to an individual.
21. The method according to the above 20, wherein the disease is any one selected from the group consisting of cancer, cardiovascular disease, neurological disorder, diabetes, autoimmune disease, inflammatory disease, viral infectious disease and allergy.

The human antibody Fc domain variant of the present invention does not bind or does not substantially bind to human FcγRs and C1q.

The human antibody Fc domain variant of the present invention does not exhibit effector functions such as antibody-dependent cellular cytotoxicity (ADCC) and antibody-dependent cellular phagocytosis (ADCP).

The human antibody Fc domain variant of the present invention possesses pH-dependent FcRn binding ability and thermal stability.

The human antibody Fc domain variant of the present invention may reduce drug toxicity and increase efficacy.

The human antibody Fc domain variant of the present invention may maintain the half-life of the drug and remove target toxicity.

### BRIEF DESCRIPTION OF THE DRAWINGS

FIGS. 1A and 1B are schematic diagrams of mammalian cell display technology of glycosylated Fc.

FIGS. 2A to 2E show results of SDS-PAGE analysis after expression and purification of highly purified tetrameric FcγRI-streptavidin and tetrameric FcγRIIIa-158V-streptavidin, FcγRI-GST, FcγRIIa-131H-GST, FcγRIIa-131R-GST, FcγRIIb-GST, FcγRIIIa-158V-GST, FcγRIIIa-158F-GST and FcRn-GST.

FIGS. 3A and 3B show results of analyzing the binding activity of fluorescently labeled tetrameric FcγRIIIa-Alexa647 and Protein A-FITC with wild type Fc (WT Fc) expressed in CHO cells.

FIG. 4 shows results of comparing amino acids by aligning the goat IgG Fc sequence and the human IgG Fc sequence.

FIGS. 5A and 5B show results of SDS-PAGE analysis of the prepared glycosylated Fc variants after expression and purification, and the mutation sequences of each glycosylated Fc variant.

FIGS. 6A and 6B show results of ELISA assay for the binding ability, i.e., binding affinity of the prepared glycosylated Fc variants to FcγRI and FcγRIIIa-158V.

FIG. 7 is a schematic diagram illustrating a site-directed mutation library for glycosylated Fc engineering.

FIG. 8 is a schematic diagram of glycosylated Fc engineering using CHO cell display.

FIG. 9 shows results of SDS-PAGE analysis after expressing and purifying the selected glycosylated Fc variants (LP1, LP5, LP6, LP7 and LP8).

FIGS. 10A to 10C show results of ELISA assay for the binding ability of glycosylated trastuzumab Fc variants introduced with the selected glycosylated Fc variants (LP1, LP5, LP7 and LP8), respectively, to FcγRI, FcγRIIa-131H, FcγRIIa-131R, FcγRIIb, FcγRIIIa-158V and FcγRIIIa-158F.

FIG. 11 shows results of SDS-PAGE analysis for a glycosylated Fc variant (LALP) with reduced binding ability to FcγRs after expression and purification, and its mutant sequence.

FIGS. 12A to 12C show results of ELISA assay for the binding ability of the glycosylated Fc variant (LALP) with reduced binding ability to FcγRs, to FcγRI, FcγRIIa-131H, FcγRIIa-131R, FcγRIIb, FcγRIIIa-158V and FcγRIIIa-158F.

FIG. 13 shows results of SDS-PAGE analysis after expression and purification of the selected glycosylated Fc variants (LALP1, LALP5, LALP6, LALP7 and LALP8), as well as their mutant sequences.

FIGS. 14A to 14C show results of ELISA assay for the binding ability of glycosylated trastuzumab Fc variants introduced with the selected glycosylated Fc variants (LALP1, LALP5, LALP6, LALP7 and LALP8), respectively, to FcγRI, FcγRIIa-131H, FcγRIIa-131R, FcγRIIb, FcγRIIIa-158V and FcγRIIIa-158F.

FIG. 15 shows results of DSF analysis for thermal stability of the discovered glycosylated trastuzumab Fc variants (LALP, LALP1, LALP5, LALP6, LALP7 and LALP8).

FIG. 16 shows results of DSF analysis for thermal stability of the discovered glycosylated trastuzumab Fc variants (LP1, LP5, LP6, LP7 and LP8).

FIG. 17 shows results of ELISA assay for FcRn binding ability of the discovered glycosylated trastuzumab Fc variants (LALP, LALP1, LALP5, LALP6 and LALP7).

FIGS. 18A and 18B shows results of ELISA assay for FcRn binding ability of the discovered glycosylated trastuzumab Fc variants (LP1, LP5 and LP6).

FIG. 19 shows results of ELIS assay for C1q binding ability of the discovered glycosylated trastuzumab Fc variants (LALP, LALP1, LALP5, LALP6 and LALP7).

FIG. 20 shows results of ELISA assay for C1q binding ability of the discovered glycosylated trastuzumab Fc variants (LPT, LP5 and LP6).

FIG. 21A shows results of SDS-PAGE analysis after expression and purification of the conventional glycosylated trastuzumab Fc variant (LALAPG), as well as its mutant sequence.

FIG. 21B shows results of comparative analysis for HER2-expressing target cell-based FcγRI binding ability of the discovered glycosylated trastuzumab Fc variants (LPT, LP5, LP6 and LP7) with LALA.

FIG. 22 shows results of analyzing the binding ability of each of the conventional glycosylated trastuzumab Fc variant (LALAPG) and the discovered glycosylated trastuzumab Fc variants (LALP1, LALP5 and LALP6) to hFcγRI.

FIG. 23 shows results of analyzing the binding ability of each of the conventional glycosylated trastuzumab Fc variant (LALAPG) and the discovered glycosylated trastuzumab Fc variants (LALP1, LALP5 and LALP6) to hFcγRIIIa-158F.

FIG. 24 shows results of SDS-PAGE analysis after expression and purification of the glycosylated trastuzumab Fc variant (LALPPG) prepared by combining P329G and the mutation discovered in the present invention, as well as its mutant sequence.

FIGS. 25A to 25C show results of ELISA assay for binding ability of the glycosylated trastuzumab Fc variants (LALP and LALPPG) discovered in the present invention to FcγRI, FcγRIIa-131H, FcγRIIa-131R, FcγRIIb, FcγRIIIa-158V and FcγRIIIa-158F, respectively.

FIG. 26 shows results of analyzing reduced PBMC-mediated ADCC function of the glycosylated trastuzumab Fc variants (LALP1, LALP5 and LALP6) discovered in the present invention using real-time cell death analysis.

FIGS. 27A and 27D show results of analyzing reduced PBMC-mediated ADCC function of the glycosylated trastuzumab Fc variants (LALP1, LALP5 and LALP6) discovered in the present invention, which were analyzed by counting the number of dead cells through real-time cell imaging.

FIG. 28 shows an analysis of the reduced NK92-mediated ADCC function of the glycosylated rituximab Fc variants (LALP1, LALP5 and LALP6) discovered in the present invention.

FIG. 29 shows results of analyzing the reduced ADCP function of the glycosylated trastuzumab Fc variants (LALP1, LALP5 and LALP6) discovered in the present invention.

FIG. 30 shows results of analyzing the reduced CDC function of the glycosylated rituximab Fc variants (LALP1, LALP5 and LALP6) discovered in the present invention.

FIGS. 31A and 31B shows results of ELISA assay for FcRn binding ability of the glycosylated trastuzumab Fc variants (LALP1, LALP5 and LALP6) discovered in the present invention in mouse, as well as in vivo PK analysis result.

FIGS. 32A and 32B show results of ELISA assay for binding ability of the glycosylated trastuzumab Fc variants (LALP1, LALP5 and LALP6) discovered in the present invention to mFcγRI, mFcγRIIb, mFcγRIII and mFcγRIV in mice, respectively.

FIGS. 33A and 33B show results of ELISA assay for binding ability of the glycosylated trastuzumab Fc variants (LALP1, LALP5 and LALP6) discovered in the present invention to cFcγRI, cFcγRIIa, cFcγRIIb and cFcγRIII in cynomolgus monkeys, respectively.

### DETAILED DESCRIPTION

The present invention provides a human antibody Fc domain variant and a use thereof.

In the present invention, there are provided human antibody Fc domain variants that do not exhibit antibody-dependent cellular cytotoxicity (ADCC) and antibody-dependent cellular phagocytosis (ADCP) because amino acid 235 of a wild-type human antibody Fc domain is substituted with proline (P), resulting in no or substantially no binding ability to human FcγRs and C1q, as well as uses of the variants as antibody-drug conjugates.

In the present invention, the usual one- and three-letter codes for natural amino acids as well as the generally acceptable three-letter codes for other amino acids such as α-aminoisobutyric acid (Aib), N-methylglycine (Sar), etc. are used.

Amino acids abbreviated in the present invention are described as follows according to the IUPAC-IUB nomenclature: alanine: A, arginine: R, asparagine: N, aspartic acid: D, cysteine: C, glutamic acid: E, glutamine: Q, glycine: G, histidine: H, isoleucine: I, leucine: L, lysine: K, methionine: M, phenylalanine: F, proline: P, serine: S, threonine: T, tryptophan: W, tyrosine: Y and valine: V.

In the present invention, the position of a specific amino acid in the amino acid sequence refers to the position numbered according to the Kabat numbering system.

As used herein, the term “amino acid modification/variation” refers to substitution, insertion and/or deletion, preferably substitution of amino acids in a polypeptide sequence. The term “amino acid substitution” or “substitution” as used herein means that an amino acid at a specific position in the polypeptide sequence of the wild-type human antibody Fc domain is replaced with another amino acid. For example, a human antibody Fc domain variant including the L235P substitution means that leucine (L), the 235th amino acid residue in the amino acid sequence of the wild-type human antibody Fc domain, is replaced with proline (P).

In the human antibody Fc domain variant of the present invention, amino acid 235 (L) of the wild-type human antibody Fc domain may be substituted with proline (P).

In one embodiment, the human antibody Fc domain variant of the present invention may include one or more amino acid substitutions selected from the group consisting of L234A, D265N, D265M, D265L, P329V, P329L, P329G and P331L in addition to L235P.

In one embodiment, the human antibody Fc domain variant of the present invention may include amino acid substitutions of L235P and L234A of the wild-type human antibody Fc domain.

In one embodiment, the human antibody Fc domain variant of the present invention may include amino acid substitutions of D265N, D265M or D265L, in addition to L235P and L234A of the wild-type human antibody Fc domain.

In one embodiment, the human antibody Fc domain variant of the present invention may include amino acid substitutions of P329V, P329L or P329G, in addition to L235P and L234A of the wild-type human antibody Fc domain.

According to an embodiment, in the human antibody Fc domain variant of the present invention, amino acid 268 of the wild-type human antibody Fc domain may be further substituted with glutamine (Q), in addition to L235P and L234A thereof.

According to an embodiment, in the human antibody Fc domain variant of the present invention, amino acid 296 of the wild-type human antibody Fc domain may be further substituted with phenylalanine (F), in addition to L235P and L234A thereof.

According to an embodiment, in the human antibody Fc domain variant of the present invention, amino acid 300 of the wild-type human antibody Fc domain may be further substituted with phenylalanine (F), in addition to L235P and L234A thereof.

According to an embodiment, in the human antibody Fc domain variant of the present invention, amino acid 333 of the wild-type human antibody Fc domain may be further substituted with valine (V), in addition to L235P and L234A thereof.

In one embodiment, the human antibody Fc domain variant of the present invention may be a human antibody Fc domain variant LALP including amino acid substitutions of L234A and L235P. The human antibody Fc domain variant LALP may include the amino acid sequence of SEQ ID NO: 1, and may be encoded with a nucleic acid molecule including the nucleotide sequence of SEQ ID NO: 2.

In one embodiment, the human antibody Fc domain variant of the present invention may be the human antibody Fc domain variant LALP1 including amino acid substitutions of L234A, L235P and D265N. The human antibody Fc domain variant LALP1 may include the amino acid sequence of SEQ ID NO: 3, and may be encoded with a nucleic acid molecule including the nucleotide sequence of SEQ ID NO: 4.

In one embodiment, the human antibody Fc domain variant of the present invention may be a human antibody Fc domain variant LALP5 including amino acid substitutions of L234A, L235P and P329V. The human antibody Fc domain variant LALP5 may include the amino acid sequence of SEQ ID NO: 5, and may be encoded with a nucleic acid molecule including the nucleotide sequence of SEQ ID NO: 6.

In one embodiment, the human antibody Fc domain variant of the present invention may be a human antibody Fc domain variant LALP6 including amino acid substitutions of L234A, L235P, and P329L. The human antibody Fc domain variant LALP6 may include the amino acid sequence of SEQ ID NO: 7, and may be encoded with a nucleic acid molecule including the nucleotide sequence of SEQ ID NO: 8.

In one embodiment, the human antibody Fc domain variant of the present invention may be a human antibody Fc domain variant LALP7 including amino acid substitutions of L234A, L235P and D265M. The human antibody Fc domain variant LALP7 may include the amino acid sequence of SEQ ID NO: 9, and may be encoded with a nucleic acid molecule including the nucleotide sequence of SEQ ID NO: 10.

In one embodiment, the human antibody Fc domain variant of the present invention may be a human antibody Fc domain variant LALP8 including amino acid substitutions of L234A, L235P, D265L and P331L. The human antibody Fc domain variant LALP8 may include the amino acid sequence of SEQ ID NO: 11, and may be encoded with a nucleic acid molecule including the nucleotide sequence of SEQ ID NO: 12.

In one embodiment, the human antibody Fc domain variant of the present invention may be a human antibody Fc domain variant LALPPG including amino acid substitutions of L234A, L235P and P329G. The human antibody Fc domain variant LALPPG may include the amino acid sequence of SEQ ID NO: 13, and may be encoded with a nucleic acid molecule including the nucleotide sequence of SEQ ID NO: 14.

In one embodiment, the human antibody (immunoglobulin) may be IgA, IgM, IgE, IgD or IgG, or variants thereof. The human antibody may be IgG1, IgG2, IgG3 or IgG4, preferably IgG1, more preferably anti-HER2 antibody, and most preferably trastuzumab. Papain digestion of antibodies forms two Fab domains and one Fc domain; in human IgG molecules, the Fc region is generated by papain digestion of the N-terminus at Cys 226.

In one embodiment, the human antibody (immunoglobulin) may be IgG1 or a modification thereof, and the Fc domain of wild-type IgG1 may include the amino acid sequence of SEQ ID NO: 15, and may be encoded with a nucleic acid molecule including the nucleotide sequence of SEQ ID NO: 16.

In one embodiment, the human antibody Fc domain variant of the present invention may have reduced binding ability to Fc gamma receptors (FcγRs) compared to the wild-type human antibody Fc domain. Here, the Fc gamma receptor may be FcγRI, FcγRIIa, FcγRIIb or FcγRIIIa.

In one embodiment, the human antibody Fc domain variant of the present invention may have reduced binding ability to C1q, compared to the wild-type human antibody Fc domain.

In one embodiment, the human antibody Fc domain variant of the present invention may have reduced effector function, compared to the wild-type human antibody Fc domain.

In one embodiment, the effector functions may be Fc-mediated effector function selected from C1q-binding, complement activation, complement dependent cytotoxicity (CDC), antibody-dependent cellular cytotoxicity (ADCC), Fc-receptor binding including Fc-gamma receptor binding, protein A-binding, protein G-binding, antibody-dependent cellular phagocytosis (ADCP), complement dependent cellular cytotoxicity (CDCC), complement-enhanced cytotoxicity, opsonization, Fc-containing polypeptide internalization, target downregulation, ADC uptake, induction of apoptosis, cell death, cell cycle arrest, and any combination thereof.

In one embodiment, the human antibody Fc domain variant of the present invention has a pH-dependent FcRn binding affinity similar to that of the wild-type human antibody Fc domain, whereby it may have an in vivo half-life and thermal stability similar to those of the wild-type human antibody Fc domain.

In one embodiment, the human antibody Fc domain variants of the present invention may be used for the purpose of not killing cells to which they bind.

In one embodiment, the human antibody Fc domain variants of the present invention may be used as an immune checkpoint inhibitor antibody or an immune cell-inducing bispecific antibody (or bispecific immune cell engaging bispecific antibody, abbrev. to “bispecific antibody”).

The human antibody Fc domain variants of the present invention may be prepared by any method known in the art. In one embodiment, the human antibody Fc domain variant according to the invention encodes a polypeptide sequence including specific amino acid modification and then, if desired, is used to form nucleic acids that are cloned into host cells, expressed and assayed. Various methods therefor have been described in Molecular Cloning—A Laboratory Manual, 3rd Ed., Maniatis, Cold Spring Harbor Laboratory Press, New York, 2001; Current Protocols in Molecular Biology, John Wiley & Sons.

Nucleic acids encoding Fc domain variants according to the invention may be inserted into expression vectors for protein expression. Expression vectors typically include proteins operably linked, i.e., placed in a functional relationship, with control or regulatory sequences, selectable markers, optional fusion partners, and/or additional elements. An Fc variant according to the present invention may be produced by a method of culturing a host cell transformed with a nucleic acid under appropriate conditions, preferably an expression vector containing a nucleic acid encoding the Fc domain variant according to the present invention, to induce protein expression. A variety of suitable host cells including mammalian cells, bacteria, insect cells and yeast may be used, but they are not limited thereto. Methods for introducing exogenous nucleic acids into host cells are known in the art and may vary depending on the host cells used. Preferably, the Fc domain variant according to the present invention may be produced using E. coli, which has high industrial value due to its low production costs, as the host cell.

The present invention provides a method for production of Fc domain variants, including the steps of: culturing a host cell into which a nucleic acid encoding an Fc domain variant is introduced under conditions suitable for protein expression; and purifying or isolating the Fc domain variant expressed from the host cell.

“FcRn” or “neonatal Fc receptor” refers to a protein that binds to the Fc region of an IgG antibody, which is encoded at least partially by the FcRn gene. FcRn may be derived from any organism including human, mouse, rat, rabbit and monkey, but it is not limited thereto.

As is known in the art, a functional FcRn protein includes two polypeptides, often referred to as light and heavy chains. The light chain is beta-2-microglobulin, and the heavy chain is encoded by the FcRn gene. Unless otherwise stated herein, FcRn or an FcRn protein refers to a complex of the FcRn heavy chain and beta-2-microglobulin.

The present invention provides an antibody including a human antibody Fc domain variant or a fragment thereof having immunological activity.

In one embodiment, an antibody or an immunologically active fragment thereof may loss its binding ability to Fc gamma receptors (FcγRs), thereby reducing its binding ability to FcγRs or C1q compared to the wild-type human antibody.

In one embodiment, the Fe gamma receptor may be FcγRI, FcγRIIa, FcγRIIb or FcγRIIIa.

In one embodiment, an antibody or an immunologically active fragment thereof may have reduced effector function compared to the wild-type human antibody.

In one embodiment, the antibody may be a polyclonal antibody, monoclonal antibody, minibody, domain antibody, bispecific antibody, antibody mimetic, chimeric antibody or antibody conjugate.

In one embodiment, human antibodies include humanized antibodies.

Fragments having immunological activity may include antibody Fab, Fd, Fab′, dAb, F(ab′), F(ab′)2, single chain fragment variable (scFv), Fv, single chain antibody, Fv dimer, and complementarity determining region or diabody.

Antibodies may be isolated or purified by various methods known in the art. Standard purification methods include chromatographic techniques, electrophoresis, immunology, precipitation, dialysis, filtration, concentration and chromatofocusing techniques. As is known in the art, a variety of natural proteins, such as bacterial proteins A, G and L, may bind to antibodies, and may be used for purification. Often, purification by specific fusion partners may be possible.

Antibodies include functional fragments of antibody molecules as well as whole antibody forms. A whole antibody has a structure of two full-length light chains and two full-length heavy chains, wherein each light chain is connected to the heavy chain by a disulfide bond. A functional fragment of an antibody molecule refers to a fragment that possesses an antigen-binding function. Examples of antibody fragments include: (i) Fab fragment consisting of a variable region (VL) of the light chain, a variable region (VH) of the heavy chain, a constant region (CL) of the light chain, and a first constant region (CH1) of the heavy chain; (ii) Fd fragment consisting of VH and CH1 domains; (iii) Fv fragment consisting of VL and VH domains of a single antibody; (iv) dAb fragment consisting of VH domain; (v) an isolated CDR region; (vi) F(ab′)2 fragment which is a bivalent fragment including two linked Fab fragments; (vii) a single chain Fv molecule (scFv) in which the VH domain and the VL domain are bound by a peptide linker to form an antigen binding site; (viii) bispecific single-chain Fv dimer (PCT/US92/09965); and (ix) diabody (WO94/13804), which is a multivalent or multispecific fragment prepared by gene fusion.

The antibody or immunologically active fragment thereof of the present invention may be selected from the group consisting of animal-derived antibodies, chimeric antibodies, humanized antibodies, human antibodies, and immunologically active fragments thereof. Antibodies may be recombinantly or synthetically produced.

Antibodies or fragments thereof having immunological activity may be isolated from the living body (not present in the living body) or non-naturally occurring, for example, may be produced synthetically or recombinantly.

The “antibody” refers to a substance produced by stimulation of an antigen within the immune system, the type of which is not particularly limited, and may be obtained naturally or unnaturally (e.g., synthetically or recombinantly). The antibodies are very stable not only in vitro but also in vivo, and have a long half-life, making them advantageous for mass expression and production. Further, an antibody inherently has a dimeric structure and thus very high avidity. A complete antibody has a structure of two full-length light chains and two full-length heavy chains, wherein each light chain is connected to the heavy chain by a disulfide bond. The constant region of an antibody is divided into a heavy chain constant region and a light chain constant region, wherein the heavy chain constant region has gamma (γ), mu (μ), alpha (α), delta (δ) and epsilon (ε) types, and is divided into subclasses such as gamma 1 (γ1), gamma 2 (γ2), gamma 3 (γ3), gamma 4 (γ4), alpha 1 (α1) and alpha 2 (α2). The constant region of the light chain has kappa (κ) and lambda (λ) types.

The “heavy chain” is interpreted to include both of: a full-length heavy chain, in which a variable region domain VH including an amino acid sequence having sufficient variable region sequence to impart specificity to an antigen, three constant region domains CH1, CH2 and CH3, and a hinge are included; and fragments thereof. Further, the “light chain” is interpreted to include both of: a full-length light chain, in which a variable region domain VL including an amino acid sequence having sufficient variable region sequence to impart specificity to an antigen and a constant region domain CL are included; and fragments thereof.

The “Fc domain,” “Fc fragment” or “Fc region” forms an antibody together with the Fab domain/fragment, and the Fab domain/fragment consists of a variable region VL of the light chain and a variable region VH of the heavy chain, a constant region CL of the light chain, and a first constant region CH1 of the heavy chain while the Fc domain/fragment consists of a second constant region CH2 and a third constant region CH3 of the heavy chain.

In one aspect, the present invention relates to a nucleic acid molecule which encodes an Fc domain variant of the present invention, an antibody including the same, or a fragment thereof having immunological activity.

In one aspect, the present invention relates to a vector including a nucleic acid molecule and a host cell including the vector.

Nucleic acid molecules of the present invention may be isolated or recombined, and may include DNA and RNA in the form of single and double chains, as well as corresponding complementary sequences. In the case of a nucleic acid isolated from a naturally occurring source, the isolated nucleic acid is a nucleic acid which is isolated from the surrounding genetic sequence present in the genome of an individual from which the nucleic acid is isolated. In the case of nucleic acids synthesized enzymatically or chemically from a template, such as PCR products, cDNA molecules or oligonucleotides, the nucleic acids resulting from these procedures may be understood as isolated nucleic acid molecules. The isolated nucleic acid molecules refer to nucleic acid molecules either in the form of separate fragments or as components of larger nucleic acid constructs. A nucleic acid is operably linked when placed in a functional relationship with another nucleic acid sequence. For example, a DNA of the pre-sequence or secretion leader is operably linked to the DNA of a polypeptide when the polypeptide is expressed as a form of preprotein before secretion, and a promoter or enhancer is operably linked to a coding sequence when the promoter or enhancer affects transcription, or a ribosome binding site is operably linked to the coding sequence when it is arranged to facilitate translation. In general, the “operably linked” means that the DNA sequences to be linked are located adjacent to each other, and in the case of the secretory leaders, it means that they are adjacent and exist within the same reading frame. However, the enhancers do not need to be located adjacent to each other. Linking is accomplished by ligation at convenient restriction enzyme sites. If such sites do not exist, synthetic oligonucleotide adapters or linkers are used according to conventional methods.

In regard to the isolated nucleic acid molecule encoding the Fc domain variant of the present invention, an antibody including the same or a fragment thereof having immunological activity, due to codon degeneracy or in consideration of preferred codon in the organism to be expressed, various modifications may be made to the coding region within the range that does not change the amino acid sequence of the Fc domain variant expressed from the coding region, the antibody including the same or a fragment thereof having immunological activity. Further, those skilled in the art will understand that, even in the portion excluding the coding region, various alternations or modifications may be made within the range that does not affect the expression of the gene, and that such modified genes are also included within the scope of the present invention. That is, as long as the nucleic acid molecule of the present invention encodes a protein having equivalent activity, one or more nucleic acid bases may be mutated by substitution, deletion, insertion or a combination thereof, and these are also included within the scope of the present invention. The sequence of such nucleic acid molecule may be single or double chain, and may be DNA molecules or RNA (mRNA) molecules.

An isolated nucleic acid molecule encoding an Fc domain variant of the present invention, an antibody including the same or a fragment thereof having immunological activity according to the present invention may be inserted into an expression vector for protein expression. The expression vectors typically include proteins operably linked, i.e., placed in a functional relationship, with control or regulatory sequences, selectable markers, optional fusion partners, and/or additional elements. Under appropriate conditions, by a method of culturing a host cell transformed with a nucleic acid, preferably, an expression vector which includes an isolated nucleic acid molecule encoding the Fc domain variant of the present invention, an antibody including the same or a fragment thereof having immunological activity thus to induce protein expression, it is possible to produce the Fc domain variant of the present invention, an antibody including the same or a fragment thereof having immunological activity. A variety of suitable host cells including mammalian cells, bacteria, insect cells and yeast, but they are not limited thereto. Methods for introducing exogenous nucleic acids into the host cells are known in the art, and will vary depending on the host cells used. Preferably, E. coli, which has high industrial value due to its low production costs, may be used to produce the Fc domain variant as the host cell.

Vectors of the present invention include plasmid vectors, cosmid vectors, bacteriophage vectors and viral vectors, etc., but they are not limited thereto. Suitable vectors include expression control elements such as promoters, operators, start codons, stop codons, polyadenylation signals and enhancers, as well as signal sequences or leader sequences for membrane targeting or secretion, and may be prepared in various ways according to the purposes. The promoter of the vector may be constitutive or inducible. The signal sequences may use: PhoA signal sequence, OmpA signal sequence, etc. When the host is Escherichia sp; α-amylase signal sequence, subtilisin signal sequence, etc. When the host is Bacillus sp.; MFα signal sequence, SUC2 signal sequence, etc. When the host is yeast; and an insulin signal sequence, α-interferon signal sequence, antibody molecule signal sequence, etc. When the host is an animal cell, but they are not limited thereto. In addition, the vector may include a selection marker for selecting host cells containing the vector, and if it is a replicable expression vector, it will include an origin of replication.

In the present invention, the “vector” refers to a transporter capable of inserting a nucleic acid sequence for introduction into a cell capable of replicating the nucleic acid sequence. The nucleic acid sequences may be exogenous or heterologous. The vectors may include plasmids, cosmids, and viruses (e.g., bacteriophages), but they are not limited thereto. Those skilled in the art may construct vectors by standard recombination techniques.

In one embodiment, when constructing the vector, expression regulatory sequences such as a promoter, terminator, enhancer, etc., sequences for membrane targeting or secretion, or the like may be appropriately selected depending on the type of the host cells to produce the Fc domain variant, an antibody including the same or a fragment thereof having immunological activity, and may be combined in various ways according to the purposes.

The “expression vector” means a vector including a nucleic acid sequence that encodes at least a portion of the gene product to be transcribed. In some cases, the RNA molecule is then translated into a protein, polypeptide, or peptide. The expression vectors may include various regulatory sequences. In addition to the regulatory sequences that regulate transcription and translation, vectors and expression vectors may also include nucleic acid sequences that also provide other functions.

The “host cell” includes eukaryotes and prokaryotes, and means any transformable organism capable of replicating the vector or expressing the gene encoded by the vector. The host cell may be transfected or transformed by the vector, which refers to a process in which an exogenous nucleic acid molecule is transferred or introduced into the host cell.

In one embodiment, the host cells may be bacteria or animal cells, wherein the animal cell line may be CHO cells, HEK cells or NSO cells, and the bacteria may be Escherichia coli.

In one aspect, the present invention relates to a fusion protein in which the Fc domain variant of the present invention, an antibody including the same or a fragment thereof having immunological activity is linked to a cargo molecule.

In one embodiment, the cargo molecule may be a detection agent, therapeutic agent, drug, peptide, growth factor, cytokine, receptor trap, chemical compound, carbohydrate moiety, enzyme, antibody or fragment thereof, DNA-based molecule, viral vector, or cytotoxic agent; one or more liposomes or nanocarriers loaded with a detection agent, therapeutic agent, drug, peptide, enzyme, antibody or fragment thereof, DNA-based molecule, viral vector or cytotoxic agent; or one or more nanoparticles, nanowires, nanotubes or quantum dots.

In one embodiment, the fusion protein may be an agonist antibody, an antagonist antibody or a therapeutic antibody.

The present invention provides an antibody-drug conjugate in which the antibody of the present invention or a fragment thereof having immunological activity is conjugated to one or more drugs.

In one embodiment, the drug may be an immune checkpoint inhibitor or a bispecific immune cell engager, and may have reduced effector function.

In one embodiment, the drug may be chimeric antigen receptor (CAR) cell therapeutics, oncolytic drug, immunotherapy agent, cytotoxic agent, angiogenesis inhibitor, kinase inhibitor, costimulatory molecule blocker, adhesion molecule blocker, anti-cytokine agent, nucleic acid therapeutics, anti-CTLA-4 agent, anti-PD-1 agent, anti-PD-L1 agent, anti-PD-L2 agent, TNF-α cross-linking agent, TRAIL cross-linking agent, anti-CD27 agent, anti-CD30 agent, anti-CD40 agent, anti-4-1BB agent, anti-GITR agent, anti-OX40 agent, anti-TRAILR1 agent, anti-TRAILR2 agent, tagretin, interferon-alpha, clobetasol, peg interferon, prednisone, romidepsin, bexarotene, methotrexate, triamcinolone cream, anti-chemokine, vorinostat, gabapentin, cyclosporine, rapamycin, FK506, detectable marker or reporter, TNF antagonist, antirheumatic agent, muscle relaxants, narcotics, non-steroidal anti-inflammatory drugs (NSAIDs), analgesics, anesthetics, sedatives, local anesthetics, neuromuscular blocker, antibacterial agent, psoriasis therapeutics, corticosteroid, anabolic steroid, erythropoietin, immunizations, immunoglobulin, immunosuppressant, growth hormone, hormone replacement drug, radiopharmaceuticals, antidepressant, antipsychotics, stimulant, asthma drug, beta agonist, inhaled steroid, epinephrine or its analogues, cytokine, cytokine antagonist, PD-1 antagonist, adenosine A2AR antagonist, CD73 inhibitor, CTLA-4 inhibitor, TIM-3 inhibitor, LAG-3 inhibitor, anthracycline, antisense oligonucleotide (ASO), siRNA, aptamer, or a combination thereof.

In one aspect, the present invention provides a pharmaceutical composition for prevention or treatment of cancer, which includes a human antibody Fc domain variant, an antibody including the same, or a fragment thereof having immunological activity, or an antibody therapeutic agent including the same as an active ingredient.

In one embodiment, the cancer may be any one selected from the group consisting of: brain tumor, melanoma, myeloma, non-small cell lung cancer, oral cancer, liver cancer, stomach cancer, colon cancer, breast cancer, lung cancer, bone cancer, pancreatic cancer, skin cancer, head or neck cancer, cervical cancer, ovarian cancer, colon cancer, small intestine. Cancer, rectal cancer, fallopian tube carcinoma, anal cancer, endometrial carcinoma, vaginal carcinoma, vulvar carcinoma, Hodgkin's disease, esophageal cancer, lymph node cancer, bladder cancer, gallbladder cancer, endocrine cancer, thyroid cancer, parathyroid cancer, adrenal cancer, soft tissue sarcoma, urethral cancer, penile cancer, prostate cancer, chronic or acute leukemia, lymphocytic lymphoma, renal or ureteral cancer, renal cell carcinoma, renal pelvic carcinoma, central nervous system tumor, primary central nervous system lymphoma, spinal cord tumor, brainstem glioma, and pituitary gland adenoma.

In one embodiment, the composition of the present invention may further include an immunogenic apoptosis inducing agent, wherein the immunogenic apoptosis inducing agent may be one or more selected from the group consisting of an anthracycline-based anticancer agent, taxane-based anticancer agent, anti-EGFR antibody, BK channel agonist, bortezomib, cardiac glycoside, cyclophosmide anticancer agent, GADD34/PP1 inhibitor, LV-tSMAC, Measles virus, bleomycin, mitoxantrone or oxaliplatin; and wherein the anthracycline-based anticancer agent may be daunorubicin, doxorubicin, epirubicin, idarubicin, pixantrone, sabarubicin or valrubicine, and the taxane-based anticancer agent may be paclitaxel or docetaxel.

The pharmaceutical composition for treatment or prevention of cancer according to the present invention may increase cancer treatment effects of conventional anticancer agents based on cancer cell killing effects by administering the composition together with chemical anticancer drugs (anticancer agents). Such administration in combination may be done simultaneously or sequentially with the anticancer agents. Examples of the anticancer agents may include: DNA alkylating agents such as mechloethamine, chlorambucil, phenylalanine, mustard, cyclophosphamide, ifosfamide, carmustine (BCNU), lomustine (CCNU), streptozotocin, busulfan, thiotepa, cisplatin and carboplatin; anti-cancer antibiotics such as dactinomycin (actinomycin D), plicamycin and mitomycin C; and plant alkaloids such as vincristine, vinblastine, etoposide, teniposide, topotecan and iridotecan, but they are not limited thereto.

The “prevention” refers to all actions that inhibit or delay the occurrence, spread and recurrence of cancer by administering the pharmaceutical composition according to the present invention.

The “treatment” refers to all actions that kill cancer cells or improve or beneficially change symptoms of cancer by administering the composition of the present invention. Anyone with ordinary knowledge in the technical field to which the present invention pertains (“those skilled in the art”) would know the exact criteria for diseases for which the composition of the present invention is effective, and determine the extent of improvement, enhancement and treatment thereof with reference to data presented by the Korean Medical Association.

The term “therapeutically effective amount” as used herein in combination with an active ingredient refers to an amount of a pharmaceutically acceptable salt of the composition effective in preventing or treating the target disease, and the therapeutically effective amount of the composition of the present invention may vary depending on several factors, for example, administration method, target site, and condition of a patient. Therefore, when used in the human body, the administration amount or dosage should be determined as a desirable amount by considering both safety and efficiency. It is also possible to estimate an amount used in humans from the effective amount determined through animal experiments. These considerations in determining the effective amount have been described in, for example, Hardman and Limbird, eds., Goodman and Gilman's The Pharmacological Basis of Therapeutics, 10th ed. (2001), Pergamon Press; and E. W. Martin ed., Remington's Pharmaceutical Sciences, 18th ed. (1990), Mack Publishing Co.

The pharmaceutical composition of the present invention is administered in a pharmaceutically effective amount. As used herein, the term “pharmaceutically effective amount” refers to an amount which is sufficient to treat a disease with a reasonable benefit/risk ratio applicable to medical treatment and does not cause side effects, and the effective dose level may be determined according to factors including health status of the patent, type and severity of cancer, activity of drug, sensitivity to the drug, administration method, administration time, administration route and excretion rate, treatment duration, drugs combined or used simultaneously, and other factors well known in the field of medicine. The composition of the present invention may be administered as an individual therapeutic agent or in combination with other therapeutic agents, may be administered sequentially or simultaneously with conventional therapeutic agents, and may be administered singly or multiple times. Considering all of the above factors, it is important to administer an amount that can achieve maximum effects with the minimum amount without side effects, and this may be easily determined by those skilled in the art.

The pharmaceutical composition of the present invention may further include pharmaceutically acceptable additives. In this case, as the pharmaceutically acceptable additives, starch, gelatinized starch, microcrystalline cellulose, lactose, povidone, colloidal silicon dioxide, calcium hydrogen phosphate, lactose, mannitol, taffy, gum arabic, pregelatinized starch, corn starch, powdered cellulose, hydroxypropyl cellulose, opadry, sodium starch glycolate, lead carnauba, synthetic aluminum silicate, stearic acid, magnesium stearate, aluminum stearate, calcium stearate, white sugar, dextrose, sorbitol, talc, or the like may be used. The pharmaceutically acceptable additive according to the present invention is preferably included in an amount of 0.1 to 90 parts by weight based on the composition, but it is not limited thereto.

The composition of the present invention may also include carriers, diluents, excipients or a combination of two or more thereof, which are commonly used in biological agents. The pharmaceutically acceptable carriers are not particularly limited as long as they are suitable for in vivo delivery of the composition, and may include, for example, the compounds described in Merck Index, 13th ed., Merck & Co. Inc., saline, sterilized water, Ringer's solution, buffered saline solution, dextrose solution, maltodextrin solution, glycerol, ethanol and one or more of these ingredients, which may be mixed and used. In addition, if necessary, other typical additives such as antioxidants, buffers, and bacteriostatic agents may be added. Further, diluents, dispersants, surfactants, binders and lubricants may be additionally added to formulate dosage forms such as aqueous solutions, suspensions, emulsions, etc., pills, capsules, granules or tablets. Furthermore, it is preferably formulated according to each disease or ingredient using an appropriate method in the art or a method disclosed in Remington's Pharmaceutical Science (Mack Publishing Company, Easton PA, 18th, 1990).

The composition of the present invention may be administered parenterally (e.g., applied intravenously, subcutaneously, intraperitoneally, or topically as an injection formulation) or orally depending on the desired method, and the dosage is determined in varied ranges by the patient's weight, age, gender, health condition, diet, administration time, administration method, excretion rate, and severity of disease. A daily dosage of the composition according to the present invention may range from 0.0001 to 10 mg/ml, preferably 0.0001 to 5 mg/ml, and more preferably administered once or several times a day.

Liquid formulations for oral administration of the composition of the present invention may include suspension, oral solution, emulsion, syrups, etc. Further, in addition to the commonly used simple diluents such as water and liquid paraffin, various excipients such as a wetting agent, sweetener, flavor, preservatives, etc. may also be included together. Formulations for parenteral administration include sterile aqueous solution, non-aqueous solvent, suspension, emulsion, lyophilized formulation, suppositories, etc.

The present invention provides a human antibody Fc domain variant; an antibody including the same or a fragment thereof having immunological activity; and an antibody-drug conjugate; alternatively, a method of treating cancer including the step of administering the above pharmaceutical composition to a subject.

In one aspect, the present invention provides a method for production of a human antibody Fc domain variant, which includes the steps of: a) culturing a host cell which includes a vector including a nucleic acid molecule encoding the human antibody Fc domain variant of the present invention; and b) recovering the polypeptide expressed by the host cell.

In one aspect, the present invention provides a method for production of an antibody with reduced effector function, which includes the steps of: a) culturing a host cell which includes a vector including a nucleic acid molecule encoding the antibody of the present invention or a fragment thereof having immunological activity; and b) purifying the antibody expressed from the host cell.

In one embodiment, purification of the antibody may include filtration, HPLC, anion exchange or cation exchange, high performance liquid chromatography (HPLC), affinity chromatography, or a combination thereof, and preferably, affinity chromatography using protein A is used.

The antibodies of the present invention or fragments thereof having immunological activity may be used for detection and imaging of targets.

The present invention provides a composition for target detection, including a conjugate in which an antibody or a fragment thereof having immunological activity is bound with a functional substance.

The targets may be antigens, antibodies, drugs, biomarkers, etc.

The condition or disease of an individual may be diagnosed through target detection and imaging.

The functional substance may be one or more selected from the group consisting of contrast agents, radioisotopes, fluorescent markers, dyes, enzymes, polypeptides, nucleic acids, carbohydrates and lipids.

The contrast agent may include lipiodol, ioxytalamic acid, iohexol, iopamidol, ioversol, iodixanol, gadobutrol, meglumine gadoterate, gadodiamide, gadobenate dimeglumine, barium sulfate, etc.

The radioisotopes may include ³H, ¹⁸F, ¹¹C, ¹⁴C, ¹⁵O, ¹³N, ³²P ³⁵S, ³⁶Cl, ⁵¹Cr, ⁵⁷Co, ⁵⁸Co, ⁵⁹Fe, ⁹⁰Y, ¹²⁵I, ¹³¹I ¹⁸⁶Re, etc.

The fluorescent marker may include green fluorescent protein (GFP), yellow fluorescent protein (YFP), blue fluorescent protein (BFP), cyan fluorescent protein (CFP), etc.

The dyes may include acridine dyes, cyanine dyes, fluorine dyes, oxazine dyes, phenanthridine dyes, rhodamine dyes, etc.

The enzyme may include β-glucuronidase, β-D-glucosidase, urease, peroxidase, alkaline phosphatase, acetylcholinesterase, glycose oxidase, hexokinase, malate dehydrogenase, glucose-6-phosphodehydrogenase, invertase, etc.

In one embodiment, the presence and progression stage of an individual's disease may be diagnosed by detecting an antigen with the composition for target detection of the present invention.

In one embodiment, the therapeutic efficacy of a drug administered to an individual may be monitored by detecting an antigen with the composition for target detection of the present invention.

The composition for target detection of the present invention may be applied to various analysis methods using immune responses. The analysis method is, for example, an immunoassay or an immunodetection method. The immunoassay or immunodetection method may include radioimmunoassay (RIA), enzyme-linked immunosorbent assay (ELISA), fluorescence immunoassay (FIA), chemiluminescence immunoassay, western blotting, etc.

The composition for target detection of the present invention may be used for in vivo detection (imaging). In vivo detection (imaging) methods may include, for example, scintigraphy, positron emission tomography (PET), single photon emission tomography (SPECT), near-infrared (NIR) optical imaging, magnetic resonance imaging (MRI), etc.

Diagnosable diseases include, for example, cancer, cardiovascular disease, neurological disorder, diabetes, autoimmune disease, inflammatory disease, viral infection, allergy and the like.

The composition for target detection of the present invention may include a reagent for performing an antigen-antibody reaction or a reagent for detecting the reaction. The reagent for performing antigen-antibody reaction includes buffers, salts, etc. The reagent for detecting the reaction includes formulations commonly used in immunoassay or immunodetection methods, for example, monoclonal antibodies, fragments thereof, or a labeled secondary antibody for recognizing conjugate, and a substrate corresponding to the label.

Hereinafter, the present invention will be described in more detail through examples.

### EXAMPLE

### Example 1. Construction of Mammalian Cell Display System for Screening Glycosylated Fc Variants

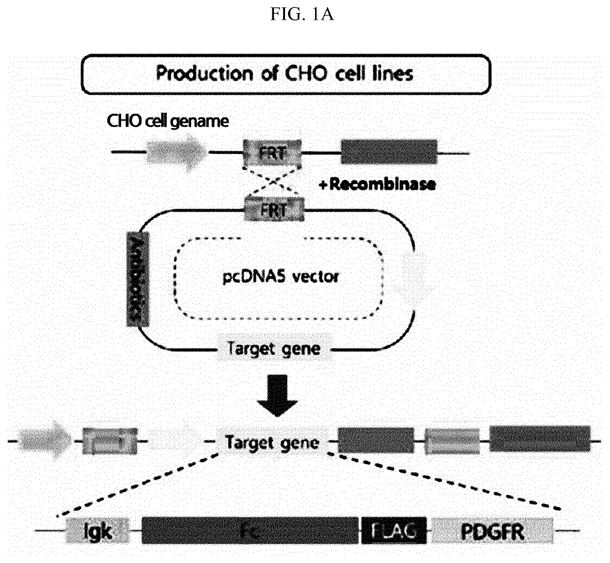

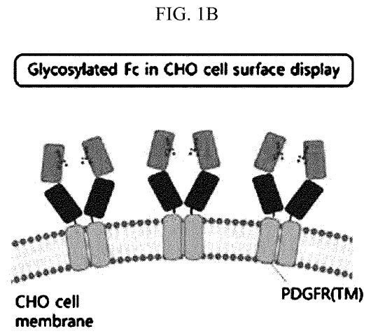

An Fc library was prepared to discover new glycosylated Fc variants that can remove immune action mechanisms. Specifically, the FLP-FRT gene recombination system used in the production of stable cell lines was utilized to display and stably screen the glycosylated Fc domain on the surface of mammalian cells. Further, in order to display the glycosylated Fc domain on the cell membrane, a platelet-derived growth factor receptor (PDGFR) transmembrane domain was fused to C-terminus of Fc. The Fc-PDGFR gene was prepared by cloning into pcDNA5/FRT plasmid (Invitrogen, V601020) into which FRT site was inserted. The corresponding plasmid induces genetic recombination by co-transfecting CHO cells (Invitrogen, R75807) into which the FRT site is inserted with the pOG44 plasmid (Invitrogen, V600520) expressing FLP, which is an enzyme causing genetic recombination, whereby CHO cell line stably expressing the glycosylated Fc through integration of Fc-PDGFR DNA into chromosomal DNA of the CHO cell was prepared. At this time, in order to select only CHO cells in which gene integration occurred after transfection, the hygromycin-B resistance gene was integrated together, further, the glycosylated Fc stable expression CHO cell lines were selected and prepared by treatment with 500 μg/ml of hygromycin-B (Invitrogen, 10687010) (FIGS. 1A and 1B).

### Example 2. FcγRs Expression and Purification

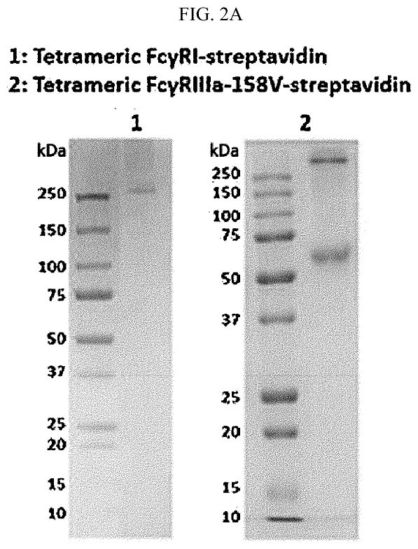

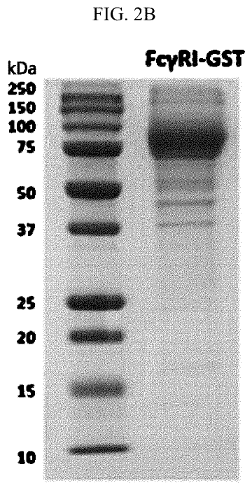

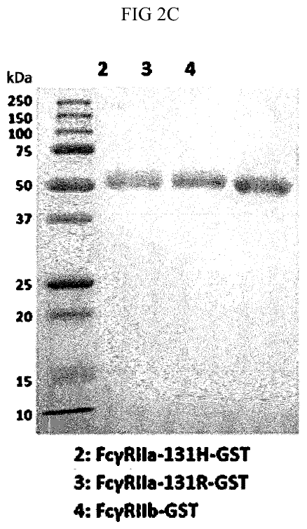

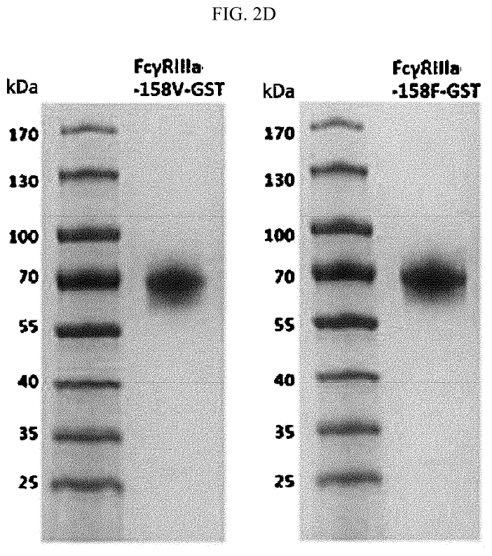

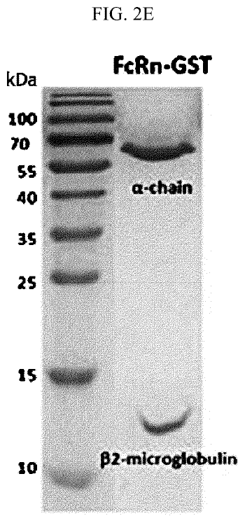

In order to conduct FACS screening of a cell line (stable cell line) stably expressing the glycosylated Fc library and select Fc variants, in which an antibody-mediated immune action mechanism was completely removed, using a CHO cell display system of the glycosylated Fc established in Example 1, Fc gamma receptors (FcγRs) were produced. Specifically, tetrameric FcγRIIIa-158V-streptavidin-His was prepared by fusing streptavidin to the C-terminus of each receptor so that efficient FACS screening would be possible by improving the visible binding affinity with Fc. Further, the tetrameric FcγRI-streptavidin-His, FcγRI-GST, FcγRIIa-131H-GST, FcγRIIa-131R-GST, FcγRIIb-GST, FcγRIIIa-158V-GST and FcγRIIIa-158F-GST, which are required to analyze the binding ability to FcγRs of the discovered glycosylated Fc variants by ELISA, were prepared and produced. Further, FcRn-GST, which is required to analyze the FcRn binding ability associated with the in vivo half-life of antibodies, was prepared and produced. In order to produce each receptor protein, each protein produced above was cloned into an animal cell expression vector, then transfected into Expi293F cells using PEI, followed by incubating for 7 days at 37° C. and 125 rpm under 8% CO₂ condition. After completion of the incubation, the supernatant was recovered, equilibrated with PBS, purified by Ni-NTA (Anti-His) or anti-GST affinity chromatography, and confirmed by SDS-PAGE gel. As a result, it was confirmed that high purity tetrameric FcγRI-streptavidin, tetrameric FcγRIIIa-158V-streptavidin, FcγRI-GST, FcγRIIa-131H-GST, FcγRIIa-131R-GST, FcγRIIb-GST, FcγRIIIa-158V-GST, FcγRIIIa-158F-GST and FcRn-GST were purified (FIGS. 2A to 2E).

### Example 3. Labeling and Validation of FcγRs

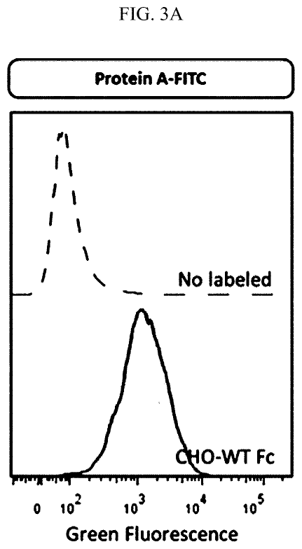

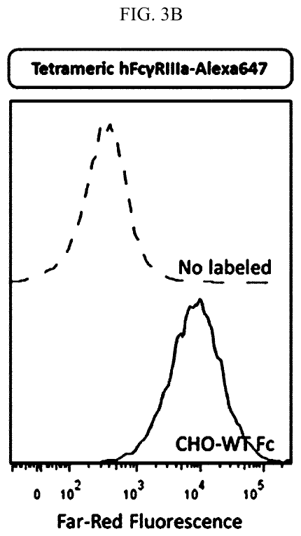

Among the produced proteins, tetrameric FcγRIIIa-streptavidin was labeled with Alexa647 (Invitrogen, A20173) fluorescent dye for FACS screening. In order to confirm whether to express or not and the expression level of each of the displayed Fc variants, Protein A (Amicogen, 1070020) whose binding sites do not overlap with FcγRs was prepared by conjugating the same with FITC (Invitrogen, F6434). At this time, conjugation of fluorescent dyes (Alexa647 and FITC) was performed according to the manual provided by each manufacturer. Through this, the fluorescence-labeled tetrameric FcγRIIIa-streptavidin-Alexa647 and Protein A-FITC were induced to bind to wild-type Fc displayed on the CHO cells, followed by confirming their activity. As a result, through fluorescence signals due to Protein A-FITC binding, it was confirmed that Protein A-FITC had normal activity and the wild-type Fc displayed on the CHO cells was stably expressed (FIG. 3A). Further, tetrameric FcγRIIIa-streptavidin-Alexa647 was also shown to normally bind to wild-type Fc (FIG. 3B), therefore, it has been verified that Alexa647 fluorescently labeled FcγRIIIa also had excellent activity, and Fcs displayed on the CHO cells were expressed normally and had their respective functions.

### Example 4. Preparation of Glycosylated Fc Variants with Reference to Goat IgG Fc Sequence

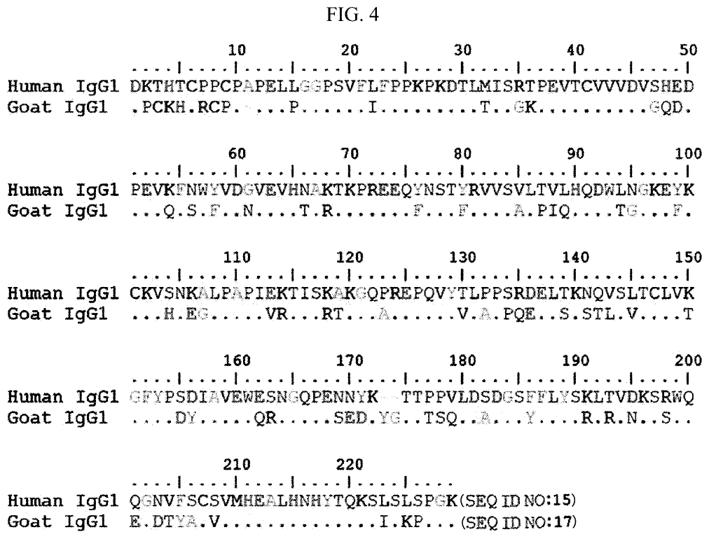

Among various animal-derived IgG antibodies, goat IgG has very low cross reactivity with human FcγRs (see S. T. Jung et al. (2010) “Aglycosylated IgG variants expressed in bacteria that selectively bind FcγRI potentiate tumor cell killing by monocyte-dendritic cells”, Proceedings of the National Academy of Sciences USA (PNAS), 107:2, 604-609; S. T. Jung, et al. (2013) “Effective phagocytosis of low Her2 tumor cell lines with engineered, aglycosylated IgG displaying high FcγRIIa affinity and selectivity”, ACS Chemical Biology, 8:2, 368-375; M. Jo et al. (2018) “Engineered aglycosylated full-length IgG Fc variants exhibiting improved FcγRIIIa binding and tumor cell clearance”, mAbs, 2:10, 278-289; H. W. Yoon et al. (2019) “Optimal combination of beneficial mutations for improved ADCC effector function of aglycosylated antibodies,” Molecular Immunology, 114, 62-71), the sequences of goat IgG Fc and human IgG Fc were compared, and mutations were introduced focusing on four regions of IgG antibody Fc (Lower hinge, B/C loop, C′/E loop and F/G loop) predicted to interact with human FcγRs, thereby preparing goat IgG Fc sequence-derived human glycosylated Fc variants (FIG. 4).

### Example 5. Animal Cell Expression and Purification of Trastuzumab Fc Variants Including Glycosylated Fc Variants

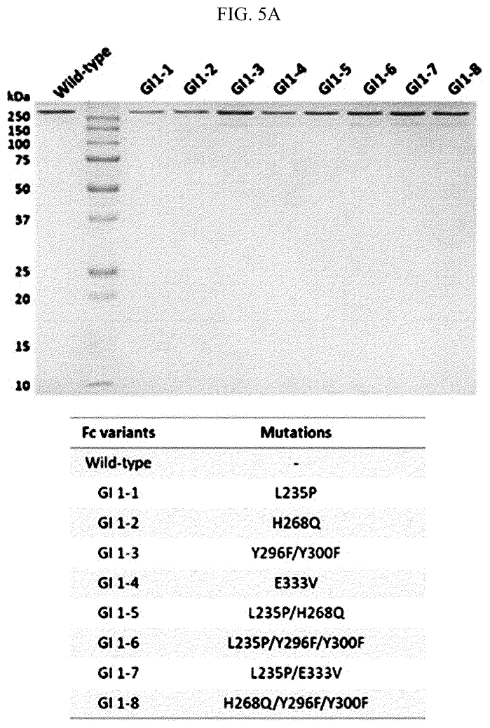

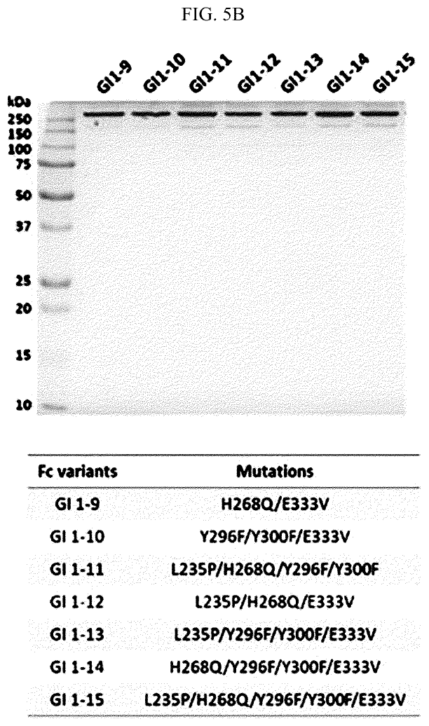

The glycosylated Fc variants prepared in Example 4 were cloned into the model antibody, that is, trastuzumab heavy chain gene to prepare an expression vector, and the heavy chain gene and light chain gene of the variants were firstly mixed at a ratio of 1:1 in Freestyle 293 expression culture medium (Gibco, 12338-018), and then PEI (polyethylenimine, Polyscience, 23966) and the expression vector gene were mixed at a ratio of 4:1, followed by incubation for 20 minutes at room temperature. Then, the mixture was transfected into Expi293F animal cells cultured at a density of 2×10⁶ cells/ml, and cultured for 7 days at 37° C. and 125 rpm under 8% CO₂ condition, followed by centrifugation to recover only the supernatant. The supernatant was equilibrated with 25×PBS and then filtered through a 0.2 μm syringe filter. Protein A resin was input into the filtered culture medium including the trastuzumab Fc variants, stirred at 4° C. for 16 hours, spun down, followed by recovering the resin and washing the same with 2 ml of 1×PBS. 300 μl of 100 mM glycine (pH 2.7) was added to the resin to elute the same, followed by neutralization using 100 μl of 1 M Tris-HCl (pH 8.0). In order to change the buffer, Amicon Ultra-4 centrifugal filter units 30K (Merck Millipore, UFC503096) were used, and highly purified glycosylated antibody trastuzumab Fc variants were ensured through SDS-PAGE analysis (FIGS. 5A and 5B).

### Example 6. Analysis of Human FcγRs Binding Ability of Glycosylated Fc Variants

ELISA assay was performed to confirm the FcγRs binding ability of the glycosylated trastuzumab Fc variants purified in Example 5 above. Specifically, 50 μl of each of FcγRs-GST (FcγRI-GST and FcγRIIIa-158V-GST) diluted with 4 μg/ml in 0.05 M Na₂CO₃ (pH 9.6) was input into a flat bottom polystyrene high bind 96 well microplate (Costar, 3590), and fixed at 4° C. for 16 hours. After blocking with 100 μl of 4% skim milk (GenomicBase, SKI400) at room temperature for 1 hour and washing 4 times with 180 μl of 0.05% PBST, 50 μl of each of the glycosylated trastuzumab Fc variants serially diluted with 1% skim milk was dispensed into each well and reacted at room temperature for 1 hour. After washing, 50 μl of HRP-Protein L (GenScript, M00098) was added, incubated at room temperature for 1 hour, and then washed. After adding 50 μl of 1-Step Ultra TMB-ELISA substrate solution (Thermo Fisher Scientific, 34028) to develop color, the reaction was terminated by adding 50 μl of 2 M H₂SO₄ at a time, and the absorbance was analyzed using an Epoch microplate spectrophotometer (BioTek).

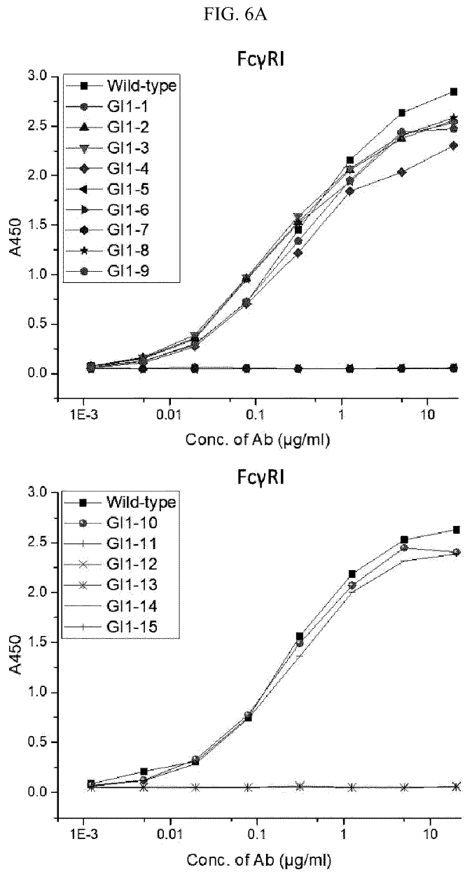

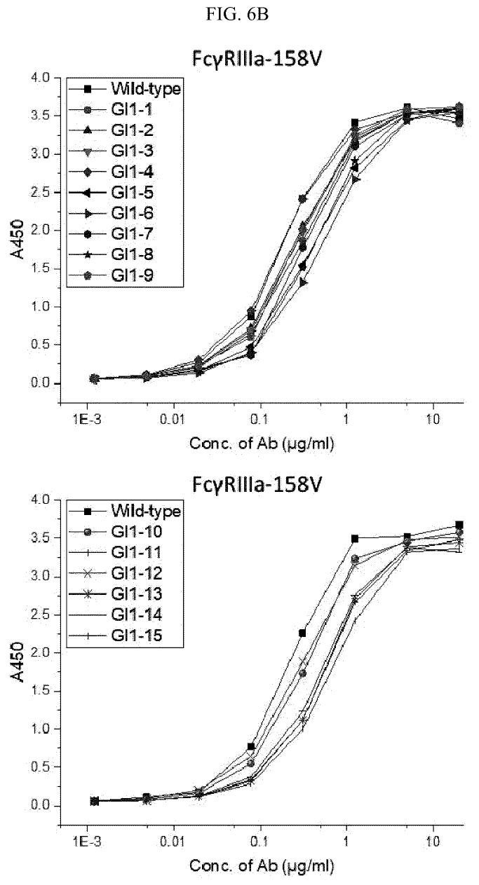

As a result, it was confirmed that, among the glycosylated Fc variants, only the Fc variants into which L235P was introduced showed that the binding ability to FcγRI (FIG. 6A) was removed, and the binding ability to FcγRIIIa-158V was reduced compared to wild-type IgG1 (FIG. 6B).

### Example 7. Construction of Glycosylated Fc Variant Library to Remove FcγRs Binding Ability

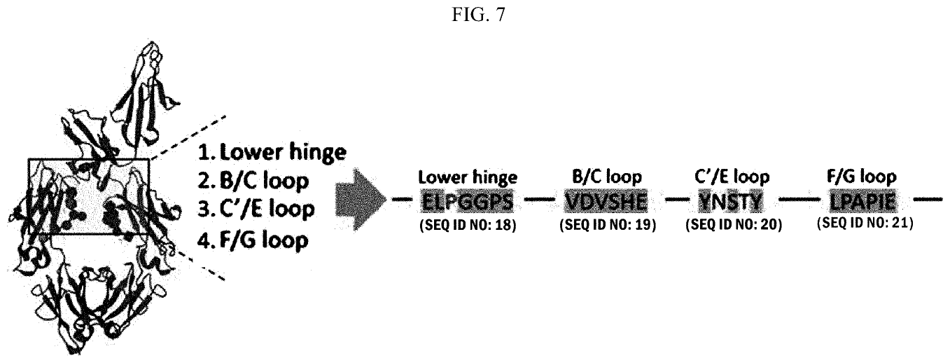

Based on the L235P variant prepared in Example 6, glycosylated Fc engineering was performed using the established CHO cell Fc display system to completely remove the binding ability to FcγRIIIa-158V. Specifically, in order to select the glycosylated Fc variants with removed FcγRs binding ability, a CHO cell-based glycosylated antibody Fc variant library was prepared, wherein mutation was introduced to four regions of Fc (Lower hinge, B/C loop, C′/E loop and F/G loop) having very important effects on FcγRs binding to human IgG1 Fc into which L235P was introduced (FIG. 7). The library gene was co-transfected with the FLP expression plasmid in the same manner as the cell display system construction method described in Example 1 above, and then subjected to a selection process using Hygromycin-B medium to prepare a glycosylated Fc variant library-stable expression CHO cell line.

### Example 8. Selection and Production of Fc Variants with Removal of FcγRs Binding Ability Using CHO Cell Display

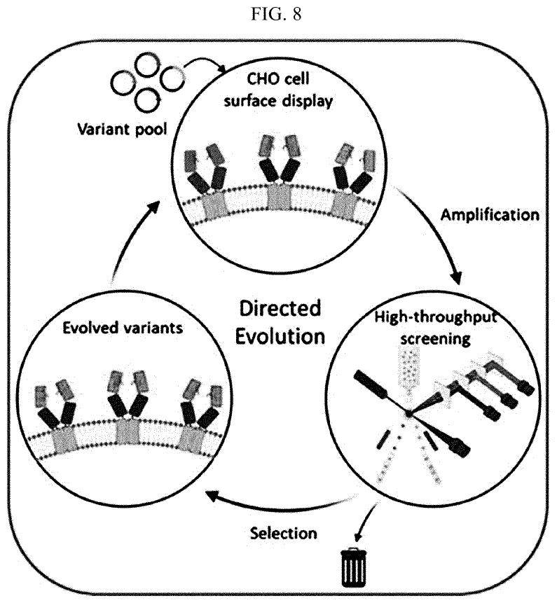

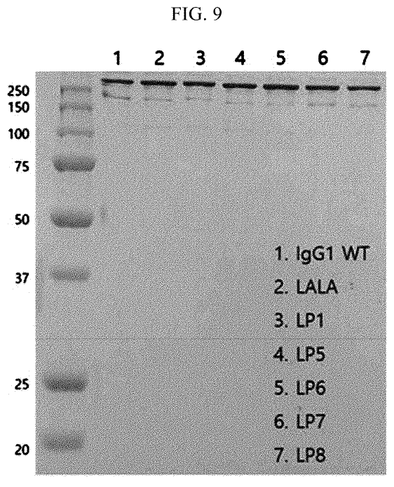

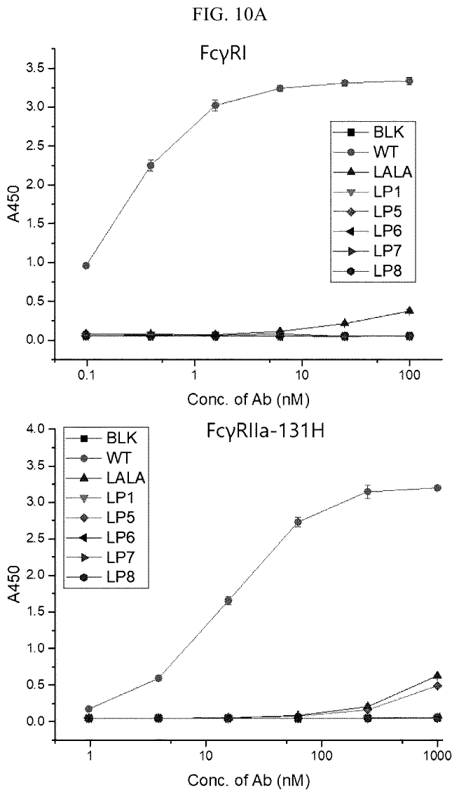

In order to select the glycosylated Fc with removed FcγRIIIa binding ability, binding of FcγRIIIa-Alexa647 and Protein A-FITC was induced in the CHO cell Fc display system and Fc library-stable expression CHO cell line established in the above example, and then the expression level by Protein A-FITC and FcγRIIIa binding were simultaneously monitored, whereby a population expected to show the removed FcγRIIIa binding ability was selected by gating through FACS screening (FIG. 8). After recovering the selected glycosylated Fc variant-expression CHO cells through genomic DNA prep, the base sequences of the selected engineered glycosylated Fc variants were confirmed (Table 1). For ELISA assay of FcγRs binding ability of the selected engineered glycosylated Fc variants (LP1, LP5, LP6, LP7 and LP8), high purity glycosylated antibody trastuzumab Fc variants were produced and purified by the method of Example 5 above (FIG. 9).

TABLE 1

| Name of variant | Introduced mutant |
|---|---|
| LP1 | L235P, D265N |
| LP5 | L235P, P329V |
| LP6 | L235P, P329L |
| LP7 | L235P, D265M |
| LP8 | L235P, D265L, P331L |

### Example 9. Analysis of Human FcγRs Binding Ability of Glycosylated Trastuzumab Fc Variants

ELISA assay was performed to confirm the FcγRs binding ability of the glycosylated trastuzumab Fc variants (LP1, LP5, LP6, LP7 and LP8) purified in Example 8 above. Specifically, 50 μl of each of FcγRs-GST (FcγRI-GST, FcγRIIa-131H-GST, FcγRIIa-131R-GST, FcγRIIb-GST, FcγRIIIa-158V-GST and FcγIIIa-158F-GST) diluted with 4 μg/ml in 0.05 M Na₂CO₃ (pH 9.6) was input into a flat bottom polystyrene high bind 96 well microplate (Costar, 3590), and fixed for 16 hours at 4° C., washed, followed by adding 100 μl of 4% skim milk (GenomicBase, SK1400) thereto and blocking for 1 hour at room temperature. The washing was performed 4 times with 180 μl of 0.05% PBST. After washing 4 times, 50 μl of each of the glycosylated trastuzumab Fc variants (LP1, LP5, LP6, LP7 and LP8) serially diluted with 1% skim milk was dispensed into each well and reacted at room temperature for 1 hour. After washing it, antibody reaction was performed using 50 μl of HRP-Protein L (GenScript, M00098) at room temperature for 1 hour, followed by washing again. After color development by adding 50 μl of 1-Step Ultra TMB-ELISA substrate solution (Thermo Fisher Scientific, 34028), the reaction was terminated by adding 50 μl of 2 M H₂SO₄, and then the absorbance was measured and analyzed using an Epoch microplate spectrophotometer (BioTek).

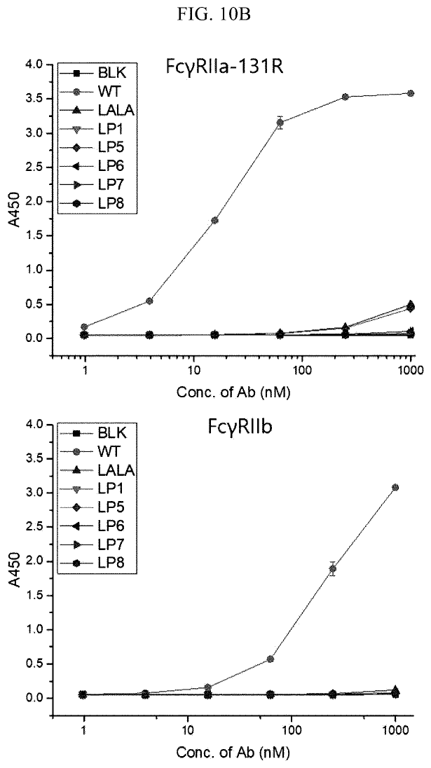

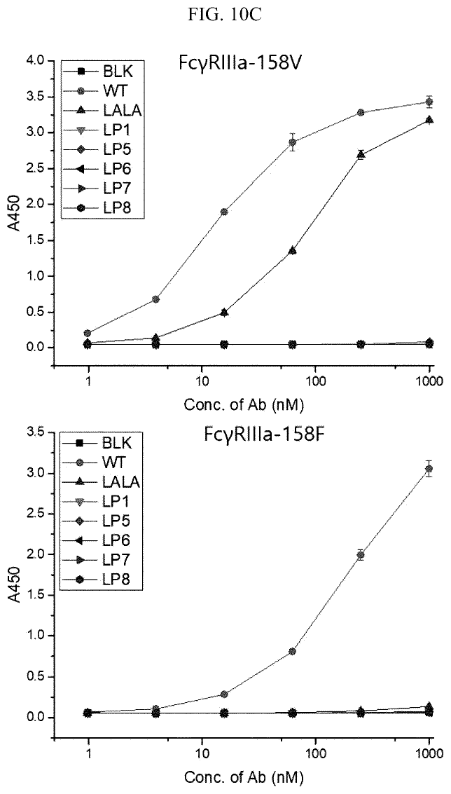

As a result, the glycosylated trastuzumab Fc variants discovered in the above example showed that, unlike the LALA (L234A/L235A) Fc variant, the binding ability to FcγRI and FcγRIIIa-158V was significantly removed. It was confirmed that the glycosylated trastuzumab Fc variants in the above examples had reduced binding ability to FcγRIIa-131H and FcγRIIa-131R than the LALA (L234A/L235A) Fc variant (FIGS. 10A to 10C).

### Example 10. Preparation of Glycosylated Fc Variants with Reduced FcγRs Binding Ability

In the above example, since the L235P variant still has binding ability to FcγRIIIa-158V, an additional mutation was introduced to reduce binding to all FcγRs. To this end, the L234A variant was combined with L235P to prepare glycosylated Fc variant LALP (L234A and L235P) (Table 2).

TABLE 2

| Name of variant | Introduced mutant |
|---|---|
| LALP | L234A, L235P |

### Example 11. Production of Glycosylated Fc Variants with Reduced FcγRs Binding Ability and Analysis of Human FcγRs Binding Ability

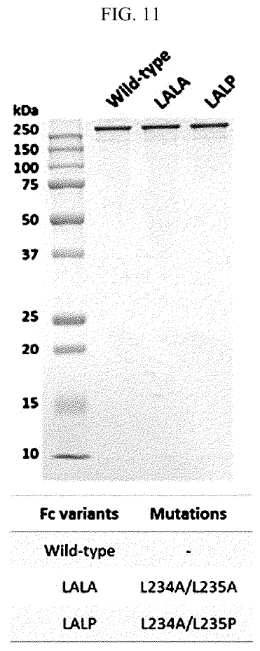

For ELISA assay of the binding ability of the glycosylated Fc variants LALP (L234A/L235P) and LALA (L234A/L235A) Fc variants prepared in Example 10 above to FcγRs, high purity glycosylated antibody trastuzumab Fc variant was produced and purified by the method of Example 5 above (FIG. 11). To confirm FcγRs binding ability of the purified glycosylated trastuzumab Fc variants, 50 μl of each of FcγRs-GST (FcγRI-GST, FcγRIIa-131H-GST, FcγRIIa-131R-GST, FcγRIIb-GST, FcγRIIIa-158V-GST and FcγIIIa-158F-GST) diluted with 4 μg/ml in 0.05 M Na₂CO₃ (pH 9.6) was input into a flat bottom polystyrene high bind 96 well microplate (Costar, 3590), and fixed for 16 hours at 4° C. After blocking with 100 μl of 4% skim milk (GenomicBase, SK1400) at room temperature for 1 hour and washing 4 times with 180 μl of 0.05% PBST, 50 μl of each of the glycosylated trastuzumab Fc variants serially diluted with 1% skim milk was dispensed into each well and reacted at room temperature for 1 hour. After washing, 50 μl of HRP-Protein L (GenScript, M00098) was added, incubated at room temperature for 1 hour, and then washed. After adding 50 μl of 1-Step Ultra TMB-ELISA substrate solution (Thermo Fisher Scientific, 34028) to develop color, the reaction was terminated by adding 50 μl of 2 M H₂SO₄ at a time, and the absorbance was analyzed using an Epoch microplate spectrophotometer (BioTek).

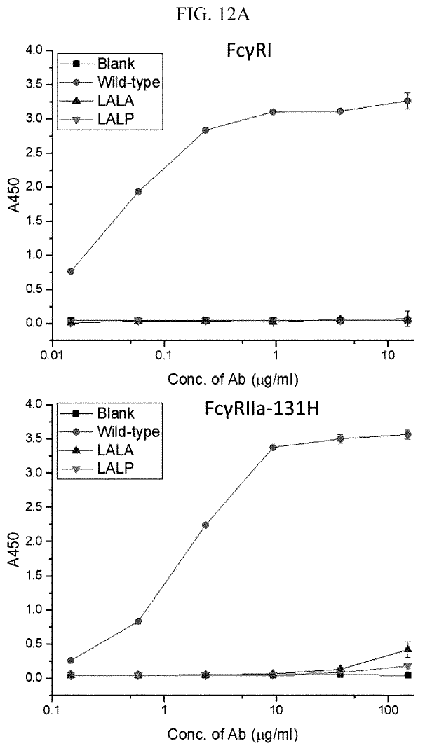

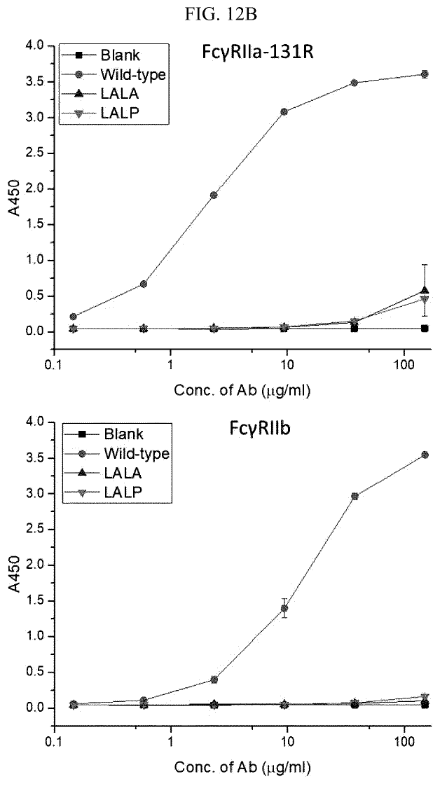

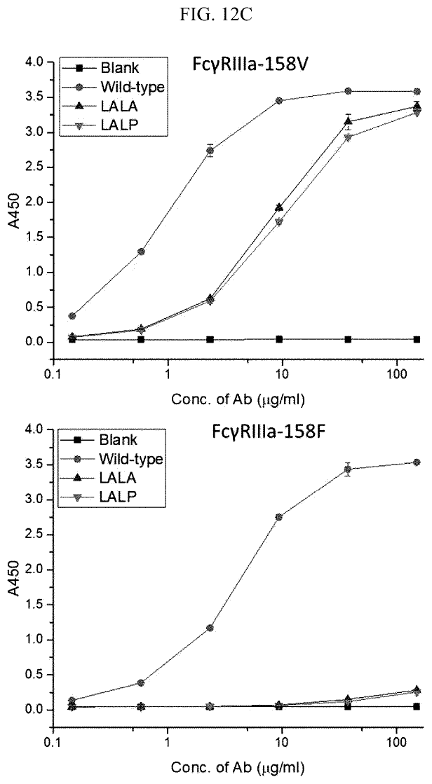

As a result, the FcγRs binding ability of the LALP (L234A/L235P) Fc variant was significantly reduced compared to that of the wild-type IgG1 Fc, and it was found that the binding ability to FcγRIIa-131H, FcγRIIa-131R and FcγRIIIa-158V was more reduced than the LALA (L234A/L235A) Fc variant, which is currently being widely used in clinical practice to reduce side effects of immune cell destruction (FIGS. 12A to 12C).

### Example 12. Construction of Glycosylated Fc Variant Library to Completely Remove FcγRs Binding Ability

The glycosylated Fc variant LALP (L234A and L235P) prepared in Example 10 above has reduced binding ability to FcγRIIIa-158V compared to the LALA (L234A/L235A) Fc variant. However, since some binding ability still remains, glycosylated Fc was engineered through the established CHO cell Fc display system to completely remove binding ability. Specifically, in order to select the glycosylated Fc variants with removed FcγRs binding ability, a CHO cell-based glycosylated antibody Fc variant library was prepared, wherein mutation was introduced to four regions of Fc (Lower hinge, B/C loop, C′/E loop and F/G loop) having very important effects on FcγRs binding to human IgG1 Fc into which LALP (L234A/L235P) was introduced (FIG. 7). The library gene was co-transfected with the FLP expression plasmid in the same manner as the cell display system construction method described in Example 1 above, and then subjected to a selection process using Hygromycin-B medium to prepare a glycosylated Fc variant library-stable expression CHO cell line.

### Example 13. Selection and Production of Fc Variants with Complete Removal of FcγRs Binding Ability Using CHO Cell Display

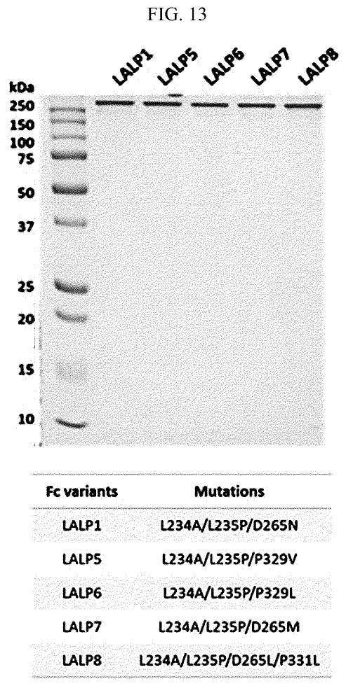

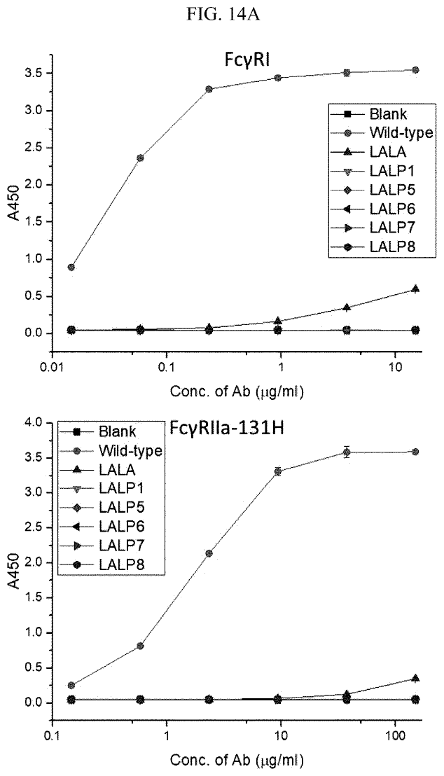

In order to select the glycosylated Fc with removed FcγRIIIa binding ability, binding of FcγRIIIa-Alexa647 and Protein A-FITC was induced in the CHO cell Fc display system and Fc library-stable expression CHO cell line established in the above example, and then the expression level by Protein A-FITC and FcγRIIIa binding were simultaneously monitored, whereby a population expected to show the removed FcγRIIIa binding ability was selected by gating through FACS screening (FIG. 8). After recovering the selected glycosylated Fc variant-expression CHO cells through genomic DNA prep, the base sequences of the selected engineered glycosylated Fc variants were confirmed (Table 3). For ELISA assay of the FcγRs binding ability of the selected engineered glycosylated Fc variants (LALP1, LALP5, LALP6, LALP7 and LALP8), high purity glycosylated antibody trastuzumab Fc variants were produced and purified by the method of Example 5 above (FIG. 13).

TABLE 3

| Name of variant | Introduced mutant |
|---|---|
| LALP1 | L234A, L235P, D265N |
| LALP5 | L234A, L235P, P329V |
| LALP6 | L234A, L235P, P329L |
| LALP7 | L234A, L235P, D265M |
| LALP8 | L234A, L235P, D265L, P331L |

### Example 14. Analysis of Human FcγRs Binding Ability of Glycosylated Trastuzumab Fc Variants with Complete Removal of FcγRs Binding Ability

ELISA assay was performed to confirm the FcγRs binding ability of the glycosylated trastuzumab Fc variants (LALP1, LALP5, LALP6, LALP7 and LALP8) purified in Example 13. Specifically, 50 μl of each of FcγRs-GST (FcγRI-GST, FcγRIIa-131H-GST, FcγRIIa-131R-GST, FcγRIIb-GST, FcγRIIIa-158V-GST and FcγIIIa-158F-GST) diluted with 4 μg/ml in 0.05 M Na₂CO₃ (pH 9.6) was input into a flat bottom polystyrene high bind 96 well microplate (Costar, 3590), and fixed for 16 hours under condition of 4° C., washed, followed by adding 100 μl of 4% skim milk (GenomicBase, SK1400) thereto and blocking for 1 hour at room temperature. The washing was performed 4 times with 180 μl of 0.05% PBST. After washing 4 times, 50 μl of each of the glycosylated trastuzumab Fc variants (LALP1, LALP5, LALP6, LALP7 and LALP8) serially diluted with 1% skim milk was dispensed into each well and reacted at room temperature for 1 hour. After washing it, antibody reaction was performed using 50 μl of HRP-Protein L (GenScript, M00098) at room temperature for 1 hour, followed by washing again. After color development by adding 50 μl of 1-Step Ultra TMB-ELISA substrate solution (Thermo Fisher Scientific, 34028), the reaction was terminated by adding 50 μl of 2 M H₂SO₄, and then the absorbance was measured and analyzed using an Epoch microplate spectrophotometer (BioTek).

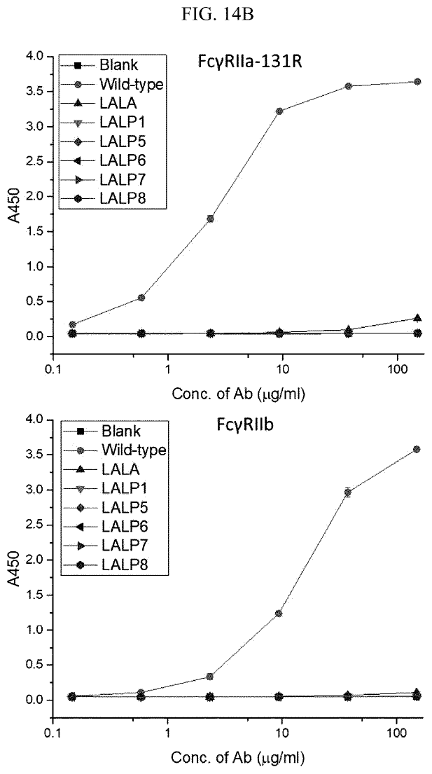

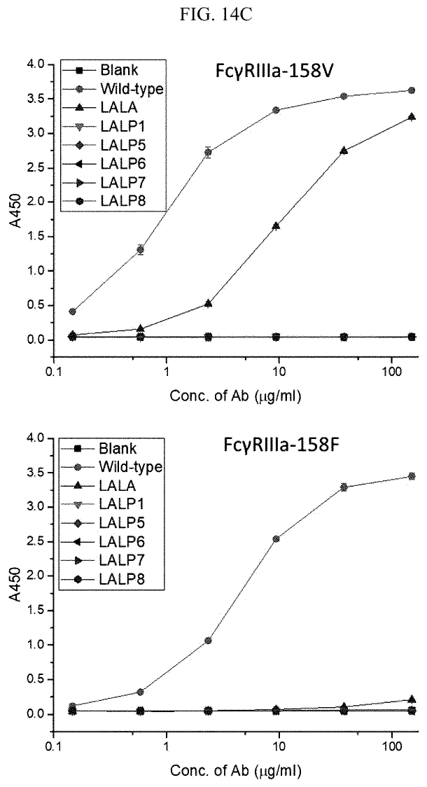

As a result, it was confirmed that the glycosylated trastuzumab Fc variants discovered in the above example had completely lost their binding ability to all FcγRs compared to wild-type IgG1 and the previously known variant LALA (L234A/L235A) (FIGS. 14A to 14C).

### Example 15. Thermal Stability Analysis of Glycosylated Trastuzumab Fc Variants With Complete Removal of FcγRs Binding Ability

To determine the temperature-dependent thermostability of glycosylated trastuzumab Fc variants (LALA, LP1, LP5, LP6, LP7, LP8, LALP, LALP1, LALP5, LALP6, LALP7 and LALP8), differential scanning fluorimetry (DSF) analysis was performed. Specifically, 45 μl of glycosylated trastuzumab Fc variant diluted with 5 μM in 1×PBS and 5 μl of SYPRO Orange (Invitrogen, S6651) dye diluted to 200× were admixed and dispensed on a PCR plate (Thermo Scientific, AB0900W). Further, as a control, 1×PBS was prepared in the same manner (all samples were performed in triplicate). An optically clear sealing film (Thermo Scientific, AB 1170) was attached to the plate containing the sample, followed by measuring fluorescence intensity while increasing the temperature by 0.03° C. per second from 25° C. to 99.9° C. using the QuantStudio 3 Real-Time PCR System (Applied Biosystems, A28567). A fluorescence value at each temperature was fitted with the Boltzmann model using OriginPro software, and then the midpoint of the sigmoidal transition curve was obtained.

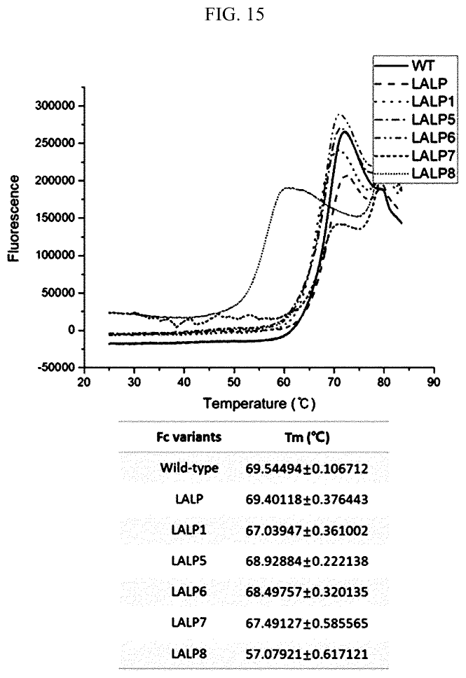

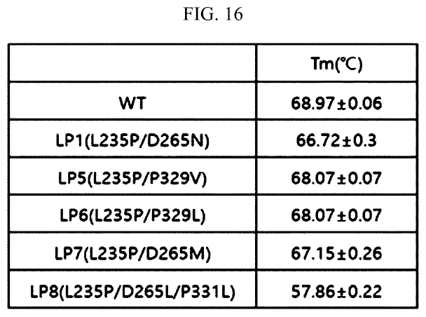

As a result, the discovered glycosylated trastuzumab Fc variants were found to have a temperature similar to the decomposition temperature of wild-type IgG1, except for the LP8 (L235P/D265L/P331L) Fc variants and the LALP8 (L234A/L235P/D265L/P331L) Fc variants (FIGS. 15 and 16).

### Example 16. Analysis of Human FcRn Binding Ability of Glycosylated Trastuzumab Fc Variants with Complete Removal of FcγRs Binding Ability

To confirm the FcRn binding ability associated with the in vivo half-life of the glycosylated trastuzumab Fc variants, ELISA assay according to pH was performed. Specifically, 50 μl of each of the glycosylated trastuzumab Fc variants diluted with 4 μg/ml in 0.05 M Na₂CO₃ (pH 9.6) was input into a flat bottom polystyrene high bind 96 well microplate (Costar, 3590), and fixed for 16 hours at 4° C., followed by adding 100 μl of 4% skim milk (GenomicBase, SK1400) thereto and blocking for 1 hour at room temperature. After washing 4 times with 180 μl of 0.05% PBST (pH 6.0/pH 7.4), 50 μl of FcRn-GST serially diluted with 1% skim milk (pH 6.0/pH 7.4) was dispensed into each well and reacted at room temperature for 1 hour. After washing, the cells were incubated for 1 hour at room temperature using 50 μl of Anti-GST-HRP Conjugate (GE Healthcare, RPN1236V) and washed again. After color development by adding 50 μl of 1-Step Ultra TMB-ELISA substrate solution (Thermo Fisher Scientific, 34028), the reaction was terminated by adding 50 μl of 2 M H₂SO₄, and then the absorbance was measured and analyzed using an Epoch microplate spectrophotometer (BioTek).

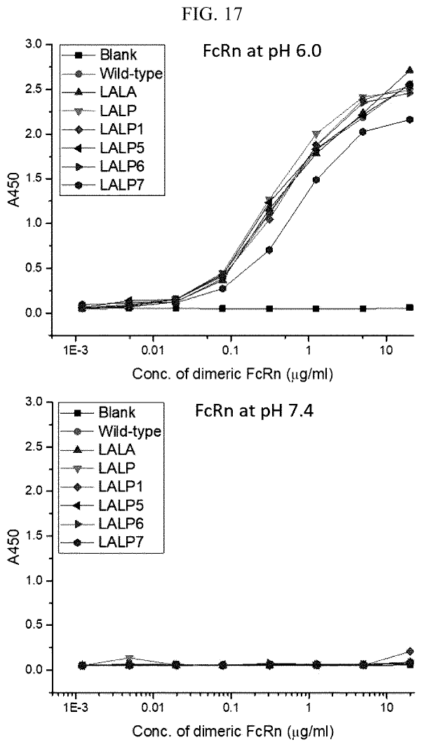

As a result, the glycosylated trastuzumab Fc variants LALP, LALP1, LALP5 and LALP6 of the present invention were found to maintain the FcRn binding properties of wild-type IgG1 (FIG. 17).

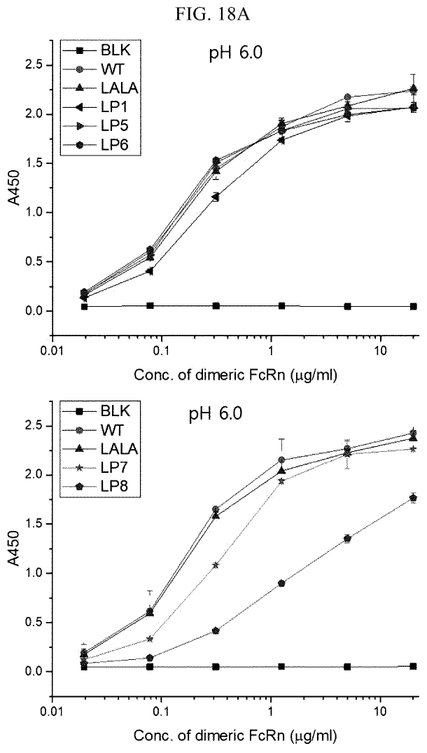

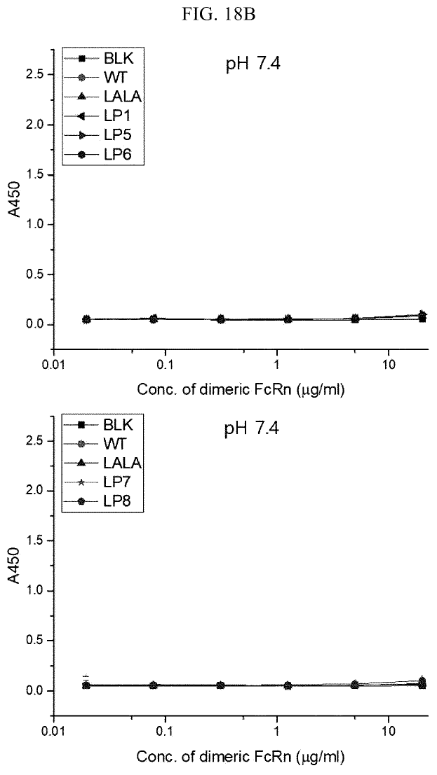

In addition, the glycosylated trastuzumab Fc variants LP1, LP5, LP6 and LP7 of the present invention were shown to maintain the FcRn binding properties of wild-type IgG1 (FIGS. 18A and 18B).

### Example 17. Analysis of Human C1q Binding Ability of Glycosylated Trastuzumab Fc Variants with Complete Removal of FcγRs Binding Ability

ELISA assay was performed to confirm the C1q binding ability of the trastuzumab Fc variants of the present invention. Specifically, 50 μl of each of the glycosylated trastuzumab Fc variants diluted with 4 μg/ml in 0.05 M Na₂CO₃ (pH 9.6) was input into a flat bottom polystyrene high bind 96 well microplate (Costar, 3590), and fixed for 16 hours at 4° C., followed by blocking with 100 μl of 4% skim milk (GenomicBase, SKI400) for 1 hour at room temperature. After washing 4 times with 180 μl of 0.05% PBST, 50 μl of C1q (Quidel, A400) protein serially diluted with 1% skim milk was dispensed into each well and reacted at room temperature for 1 hour. After washing the plate, 50 μl of anti-C1q-HRP (Invitrogen, PA1-84324) was added to each well, incubated at room temperature for 1 hour, and washed again. After color development by adding 50 μl of 1-Step Ultra TMB-ELISA substrate solution (Thermo Fisher Scientific, 34028), the reaction was terminated by adding 50 μl of 2 M H₂SO₄, and then the absorbance was measured and analyzed using an Epoch microplate spectrophotometer (BioTek).

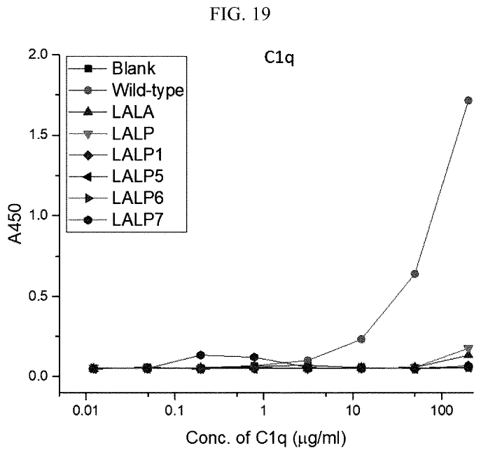

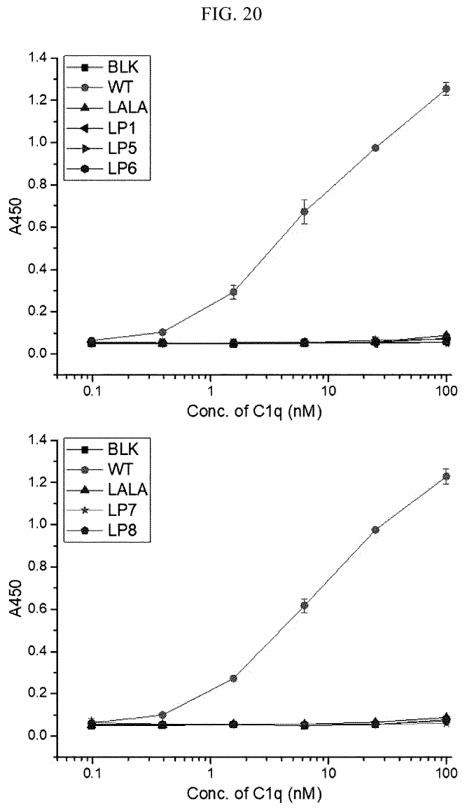

As a result, all of the discovered glycosylated trastuzumab Fc variants (LALP, LALP1, LALP5, LALP6 and LALP7) were found to have their binding ability to C1q effectively removed (FIG. 19).

In addition, all of LP1, LP5, LP6, LP7 and LP8 were confirmed to have their binding ability to C1q effectively removed (FIG. 20).

### Example 18. Analysis of Human FcγRs Binding Ability of Glycosylated Trastuzumab Fc Variants with Complete Removal of FcγRs Binding Ability

### 18-1. Preparation and Production of Control Glycosylated Trastuzumab Fc Variants

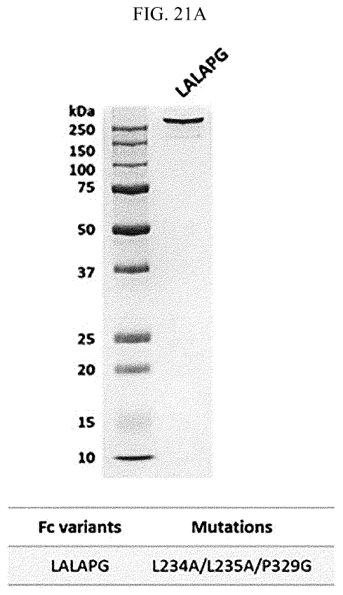

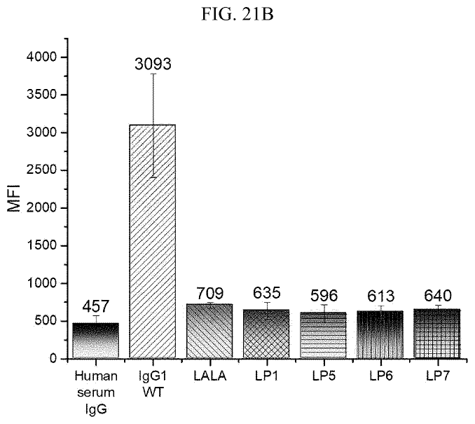

LALAPG (L234A/L235A/P329G) (Reference: WO2012130831A1) Fc variants currently in clinical development were prepared to compare the binding ability to FcγRs with the glycosylated Fc variants of the present invention, and these LALAPG (L234A/L235A/P329G) Fc variants were produced and purified by the method of Example 5 above (FIG. 21A).

### 18-2. Analysis of Human FcγRI Binding Ability Based on HER2-Expressing Target Cells

In order to confirm and compare FcγRI binding affinities between the glycosylated trastuzumab Fc variants (LP1, LP5, LP6 and LP7) and LALA prepared in Example 18-1 above by reproducing the in vivo mechanism of action, binding ability analysis was performed based on SKOV-3 cells that express HER2 as the target antigen of the trastuzumab Fc variants. Specifically, 4×10⁶ SKOV-3 cells were treated with 100 nM each of trastuzumab WT, trastuzumab LALA variant, and the glycosylated trastuzumab Fc variants (LP1, LP5, LP6 and LP7) of the present invention, the binding between 50 nM of tetrameric FcγRI-Alexa647 and Protein A-FITC at a ratio of 1:1000 or the binding between 100 nM of tetrameric FcγRI-Alexa647 and Protein A-FITC at a ratio of 1:1000 was induced, followed by incubation for 1 hour. After washing with 1×PBS/EDTA, the expression level by Protein A-FITC and the removed FcγRI binding ability were analyzed using FACS.

As a result, it was confirmed that the glycosylated trastuzumab Fc variants of the present invention not only have a lower FcγRI binding ability than the conventional Fc variant LALA (L234A/L235A), but also have the binding intensity similar to that of human serum IgG used as a negative control (FIG. 21B).

### 18-3. Analysis of Human FcγRs Binding Ability Using Octet

In order to confirm and compare FcγRs binding affinities of the glycosylated trastuzumab Fc variants (LALP1, LALP5 and LALP6) and LALAPG prepared in Example 18-1 above by reproducing the in vivo mechanism of action, analysis was performed using octet.

Octet is a bio-layer interferometry (BLI) system that measures bonds between biological molecules by applying the interference phenomenon of light, and compares a difference in interference patterns of white light reflected from the surface in which a ligand is bound to the surface of the biocenter tip, and the surface in which the ligand is not bound to the same. In other words, the binding or dissociation phenomenon of two molecules was measured and the binding difference between the molecules was analyzed.

Specifically, an Fc variant-introduced trastuzumab model antibody diluted in 1× kinetic buffer or 1×PBS was loaded onto the FAB2G sensor (a sensor chip that binds to the CH1 domain of human IgG). Then, monomeric FcγRs diluted in 1× kinetic buffer or 1×PBS were flowed at different concentrations to measure the affinity between FcγR and antibodies. When analyzing FcγRI, the antibody and FcγRI were diluted in 1× kinetic buffer, wherein FcγRI was diluted two-fold from 500 nM and analyzed to a concentration of 62.5 nM. When analyzing other FcγRs, both antibodies and FcγRs were diluted in 1×PBS, wherein FcγRs were diluted two-fold from 5000 nM and analyzed to a concentration of 625 nM.

The analysis results were confirmed through the difference in the interference patterns of white light when R_{eq} (the calculated response at equilibrium) signal was present. R_{eq} is a signal when rates of binding and dissociation of two molecules are balanced, and the difference in the interference patterns of white light when the R_{eq} signal is present was indicated as Binding (nm) on the Y axis.

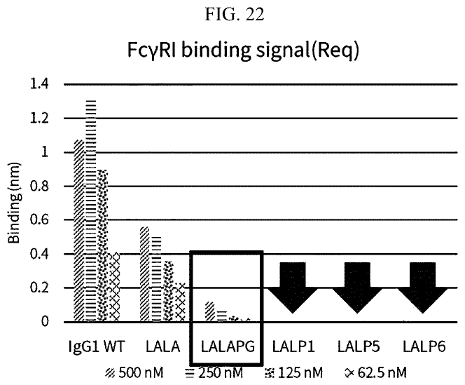

As a result, the trastuzumab Fc variants of the present invention not only had lower FcγRI binding ability than the conventional Fc variant LALA (L234A/L235A), but also had lower FcγRI binding ability than LALAPG (L234A/L235A/P329G). It was confirmed that the inventive variants exhibit excellent effects of completely removed binding ability to hFcγRI (FIG. 22).

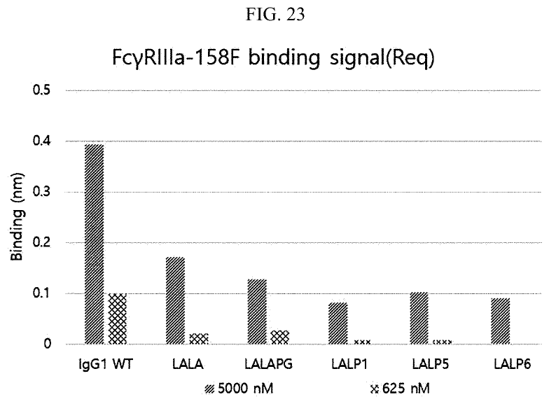

Further, it was confirmed that the trastuzumab Fc variants of the present invention have significantly reduced binding ability to FcγRIIIa-158F, whereas LALA (L234A/L235A) and LALAPG (L234A/L235A/P329G) still have strong binding ability to the same (FIG. 23).

The trastuzumab Fc variants of the present invention have binding ability to FcγRI, FcγRIIa-131H, FcγRIIa-131R, FcγRIIb, FcγRIIIa-158V and FcγRIIIa-158F completely removed. In particular, even with regard to FcγRI and FcγRIIIa-158F, to which the LALAPG variant has the binding ability, the trastuzumab Fc variants of the present invention were confirmed to have the binding ability completely removed (Table 4).

TABLE 4

| FcγRI | | | FcγRIIa-131H | | | FcγRIIa-131R | | | |
|---|---|---|---|---|---|---|---|---|---|
| | kₒₙ | k_{dis} | | kₒₙ | k_{dis} | | kₒₙ | k_{dis} | FcγRIIb |
| K_{D} | (10⁴/ | (10⁻³/ | K_{D} | (10⁴/ | (10⁻³/ | K_{D} | (10⁴/ | (10⁻³/ | K_{D} |
| (nM) | Ms [symbol] | s) | (nM) | Ms) | s) | (nM) | Ms) | s) | (nM) |

| Trastuzumab | 1.41 ± | 8.96 ± | 0.126 ± | 610 ± | Steady- | 790 ± | Steady- | 3600 ± |
|---|---|---|---|---|---|---|---|---|
| WT | 0.04 | 0.0927 | 0.0033 | 42 | state | 22 | state | 66 |
| Trastuzumab- | 136 ± | 14.9 ± | 20.3 ± | 38000 ± | Steady- | 22000 ± | Steady- | 390000 |
| LALA | 5.19 | 0.508 | 0.35 | 46000 | state | 5300 | state | |
| Trastuzumab- | w.b | w.b | w.b | n.b | n.b | n.b | n.b | n.b |
| LALAPG | | | | | | | | |
| Trastuzumab- | n.b | n.b | n.b | n.b | n.b | n.b | n.b | n.b |
| LALP1 | | | | | | | | |
| Trastuzumab- | n.b | n.b | n.b | n.b | n.b | n.b | n.b | n.b |
| LALP5 | | | | | | | | |
| Trastuzumab- | n.b | n.b | n.b | n.b | n.b | n.b | n.b | n.b |
| LALP6 | | | | | | | | |

| FcγRIIb | | FcγRIIIa-158V | | | FcγRIIIa-158F | | |
|---|---|---|---|---|---|---|---|
| kₒₙ | k_{dis} | | kₒₙ | k_{dis} | | kₒₙ | k_{dis} |
| (10⁴/ | (10⁻³/ | K_{D} | (10⁴/ | (10⁻³/ | K_{D} | (10⁴/ | (10⁻³/ |
| Ms) | s) | (nM) | Ms) | s) | (nM) | Ms) | s) |

| Trastuzumab | Steady- | 340 ± | 11.3 ± | 38.3 ± | 679 ± | 9.3 ± | 63.2 ± |
|---|---|---|---|---|---|---|---|
| WT | state | 18 | 0.371 | 1.56 | 54.6 | 0.578 | 3.22 |
| Trastuzumab- | Steady- | 1190 ± | 7.02 ± | 83.3 ± | 1360 ± | 2.43 ± | 33 ± |
| LALA | state | 89.4 | 0.445 | 3.39 | 16.7 | 0.192 | 3.11 |
| Trastuzumab- | n.b | n.b | n.b | n.b | w.b | w.b | w.b |
| LALAPG | | | | | | | |
| Trastuzumab- | n.b | n.b | n.b | n.b | n.b | n.b | n.b |
| LALP1 | | | | | | | |
| Trastuzumab- | n.b | n.b | n.b | n.b | n.b | n.b | n.b |
| LALP5 | | | | | | | |
| Trastuzumab- | n.b | n.b | n.b | n.b | n.b | n.b | n.b |
| LALP6 | | | | | | | |

[symbol]

indicates data missing or illegible when filed

### Example 19. Production of Combinatorial Glycosylated Fc Variants and Analysis Of Human FcγRs Binding Ability

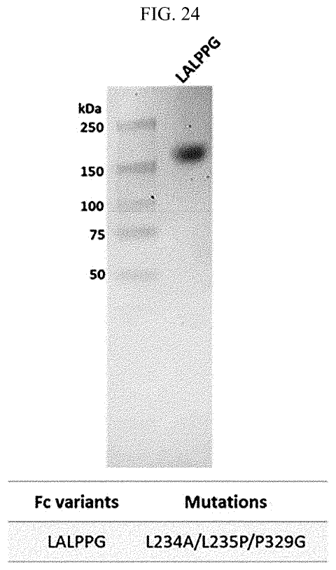

To analyze whether combining the known P329G mutation with the LALP (L234A/L235P) mutation of the present invention has effects on reduction in FcγRs binding, LALPPG (L234A/L235P/P329G) Fc variant was prepared and produced (Table 5). Afterwards, the trastuzumab LALPPG variant was produced and purified by the method of Example 5 above (FIG. 24). ELISA assay was performed to compare FcγRs binding abilities of the purified trastuzumab LALPPG variant and trastuzumab LALP variant with the conventional trastuzumab LALA variant and trastuzumab LALAPG variant.

Specifically, 50 μl of each of FcγRs-GST (FcγRI-GST, FcγRIIa-131H-GST, FcγRIIa-131R-GST, FcγRIIb-GST, FcγRIIIa-158V-GST and IIIa-158F-GST) diluted with 4 μg/ml in 0.05 M Na₂CO₃ (pH 9.6) was input into a flat bottom polystyrene high bind 96 well microplate (Costar, 3590), and fixed for 16 hours under condition of 4° C., followed by adding 100 μl of 4% skim milk (GenomicBase, SK1400) thereto and blocking for 1 hour at room temperature. The washing was performed 4 times with 180 μl of 0.05% PBST. After washing 4 times, 50 μl of each of the glycosylated trastuzumab Fc variants serially diluted with 1% skim milk was dispensed into each well and reacted at room temperature for 1 hour. After washing it, incubation was performed by adding 50 μl of HRP-Protein L (GenScript, M00098) at room temperature for 1 hour, followed by washing again. After color development by adding 50 μl of 1-Step Ultra TMB-ELISA substrate solution (Thermo Fisher Scientific, 34028), the reaction was terminated by adding 50 μl of 2 M H₂SO₄, and then the absorbance was measured and analyzed using an Epoch microplate spectrophotometer (BioTek).

TABLE 5

| Name of variant | Introduced mutant |
|---|---|
| LALPPG | L234A, L235P, P329G |

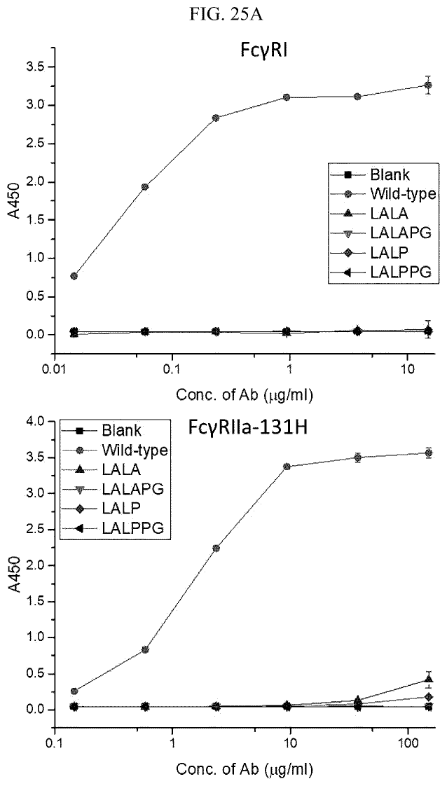

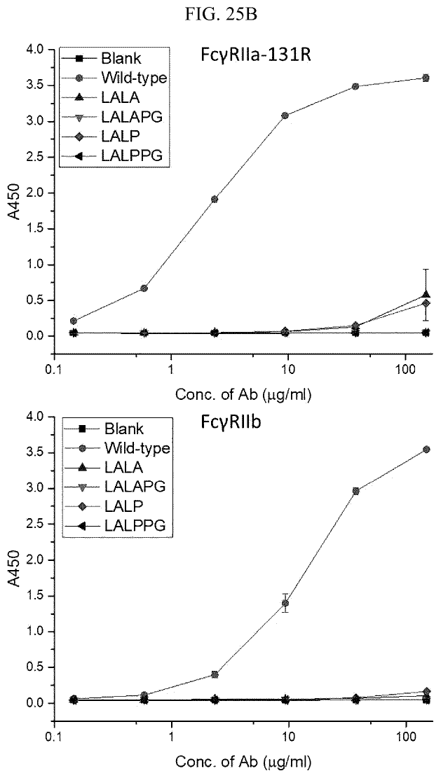

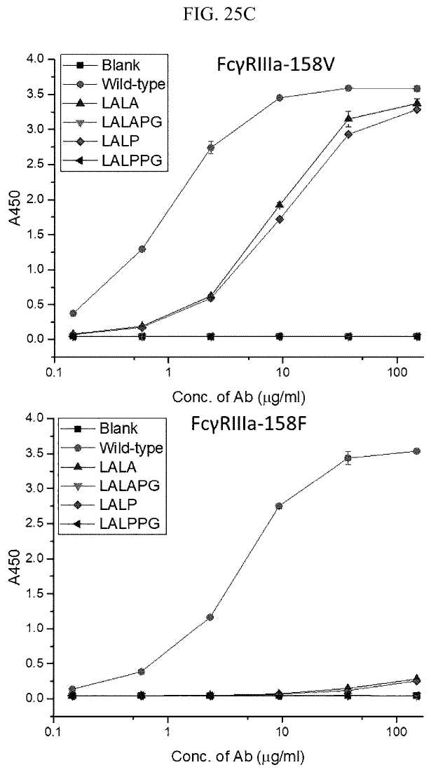

As a result, it was shown that the LALPPG (L234A/L235P/P329G) Fc variant did not bind to all FcγRs (FIGS. 25A to 25C). Through this, it was confirmed that the LALP (L234A/L235P) Fc variants may have FcγRs binding ability effectively removed even in combination with the known P329G variant.

### Example 20. Confirmation of PBMC-Mediated ADCC Removal of Glycosylated Trastuzumab Fc Variants

The reduced effector function of the glycosylated Fc variants of the present invention was confirmed. Real-time cell analysis was used to analyze human peripheral blood mononuclear cell (hPBMC)-mediated antibody-dependent cellular cytotoxicity (ADCC) of the glycosylated Fc variants. Electric current flows through a cell culture vessel with a gold sensor attached to the bottom surface, and if attached cells cover the surface of the sensor, a resistance is generated and less current is generated. However, if there are no cells or the cells have been dead, the resistance decreases while the current is generated frequently. Utilizing this fact, cell death was determined. Based on SKBR-3 cells that express HER2, the target antigen of trastuzumab Fc variants, cell death analysis was performed. 10,000 cells/well of SKBR-3 in RPMI (Gibco, 11875093) containing 10% FBS (Gibco, 16000044) were seeded on E-Plate (Agilent, 300600900) and then input into xCELLigence RTCA SP (Agilent, 300600900), followed by growing for cell adhesion and proliferation. Thereafter, PBMCs were isolated from whole blood obtained from a healthy donor using Histopaque 1077 (Sigma Aldrich, 10771), washed with 1×PBS, and then diluted with RPMI medium mixed with 10% FBS. PBMC and antibodies were placed in the E-plate, and were left at room temperature for 30 minutes to allow the cells to settle. The E-plate was placed in the RTCA equipment and monitored in real time through RTCA Software 1.2 (Agilent) over time at 37° C. and 5% CO₂, and cytolysis was analyzed according to the time point. Cells killed through hPBMC and antibodies were analyzed for a decrease in resistance value, and the results were expressed as cytolysis (%) using the equation below.

$$Cytolysis ( \% ) = ( Resistance value of well administered only target cells - Resistance value of sample well ) / Resistance value of well administered only target cells \times 100$$

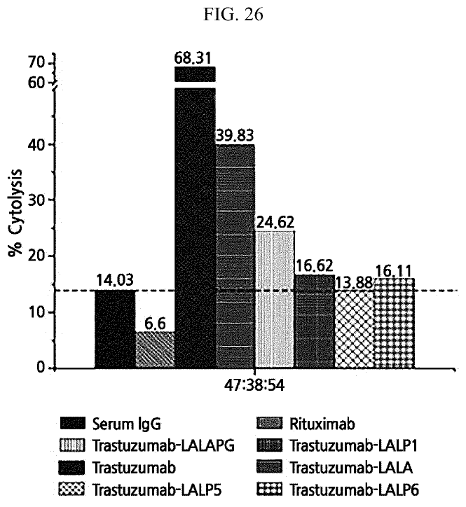

As a result, the glycosylated trastuzumab Fc variants of the present invention showed a significantly lower cytolysis rate than the conventional Fc variant LALA (L234A/L235A), thereby indicating that ADCC activity was exceptionally reduced. It was confirmed that ADCC was removed more effectively than the LALAPG (L234A/L235A/P329G) Fc variants (FIG. 26).

Further, cell death caused by ADCC was confirmed through real-time cell imaging. SKBR-3 was mixed with 2 μM Red CMTPX Dye (Invitrogen, C34552) diluted in no phenol red RPMI-1640 (Gibco, 11835030) medium and incubated for 30 minutes at 37° C. and 5% CO₂. After labeling, cells were washed with 1×PBS, and then 1×10⁴ cells/well of SKBR-3 were placed on a black plate (Greiner, 655090), followed by overnight incubation using no phenol red RPMI media containing 10% FBS and 1× Antibiotic-Antimycotic (Gibco, 15240062) in 5% CO₂ incubator. The next day, PBMC were isolated from whole blood obtained from a healthy donor using Histopaque 1077. PBMCs were labeled with 0.5 μg/ml Hoechst 33342 (Invitrogen, H3570) diluted in 1×PBS for 5 minutes at room temperature, and washed three times with 1×PBS. After treating each well with PBMC 1×10⁵ cells/well, antibodies and Caspase-3/7 Detection Reagents (Invitrogen, C10423), the plate was placed in LionheartFX (Agilent) equipment, and cell death was imaged under a fluorescence microscope over time at 37° C. and 5% CO₂.

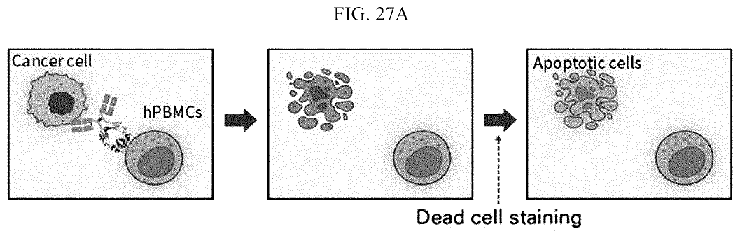

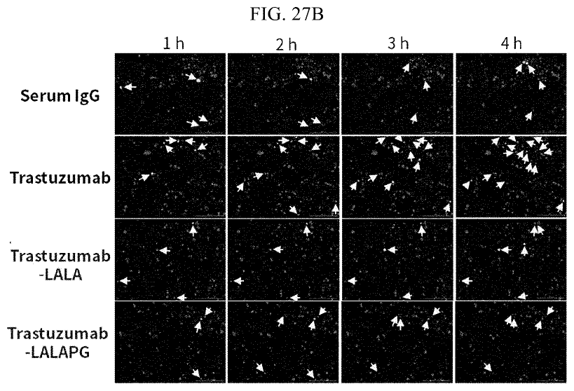

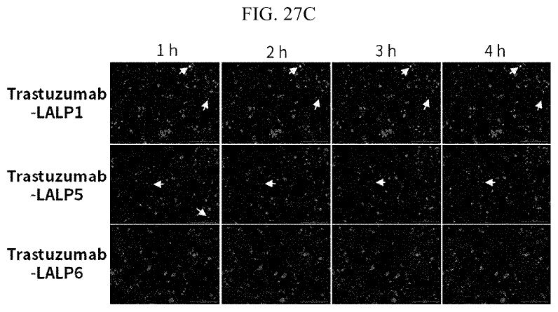

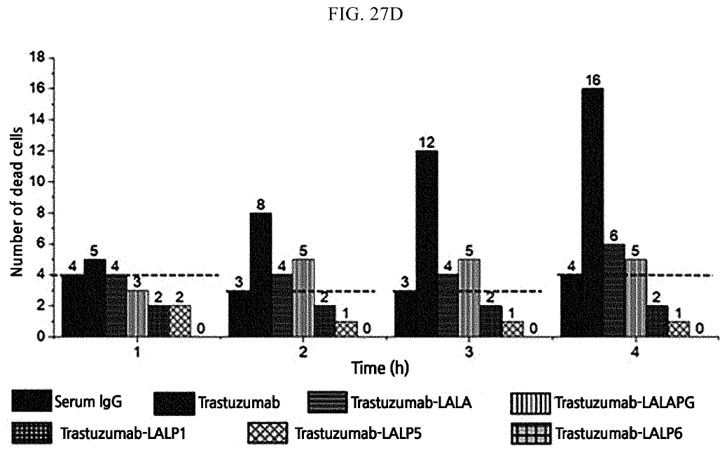

When apoptosis of cancer cells occurs through human peripheral blood mononuclear cells (hPBMCs), such apoptosis was imaged as green dots, and the activity of ADCC is confirmed by counting the green dots (FIGS. 27A to 27C).

As a result, the glycosylated trastuzumab Fc variants of the present invention killed significantly fewer cells than the conventional Fc variant LALA (L234A/L235A). It was confirmed that the glycosylated trastuzumab Fc variants of the present invention killed a smaller number of cells, even compared to LALAPG (L234A/L235A/P329G) Fc variants, thereby resulting in reduction of ADCC activity (FIG. 27D).

### Example 21. Confirmation of NK92-Mediated ADCC Removal of Glycosylated Rituximab Fc Variants

The glycosylated Fc variants of the present invention were introduced into a rituximab model antibody and NK92-mediated antibody-dependent cellular cytotoxicity (ADCC) was analyzed. Ramos, a cancer cell line expressing the target antigen of rituximab, and NK92 cell line (ATCC, PTA8836) expressing GFP-FcγRIIIa were recovered and then cell downed at 300×g for 3 minutes and 125×g for 10 minutes, respectively, followed by resuspension with PBS and washing. The prepared target cells and effector cells were input into a V-bottom 96-well plate (Corning, 3894) in amounts of 20,000 cells/well of Ramos, 200,000 cells/well (E:T=10:1) of GFP-FcγRIIIa-NK92, together with antibody samples, and then were incubated in 5% CO₂ incubator for 3 hours at 37° C. to induce ADCC reaction. After incubation, 5 nM of Sytox Red (Invitrogen, S34859) dye was added, followed by incubation at room temperature for 15 minutes and staining dead cells. After the reaction was completed, the plate was spun down at 200×g for 5 minutes to recover pellets, the pellets were washed twice with PBS and analyzed using BD FACS Lyric (BioTek) equipment.

After staining the dead cells using NK92, the number of dead cells was analyzed by FACS.

The results were expressed as cytotoxicity (%) using the equation below.

$$Cytotoxicity ( \% ) = ( Number of dead cells / Number of total cells ) \times 100 - Background$$

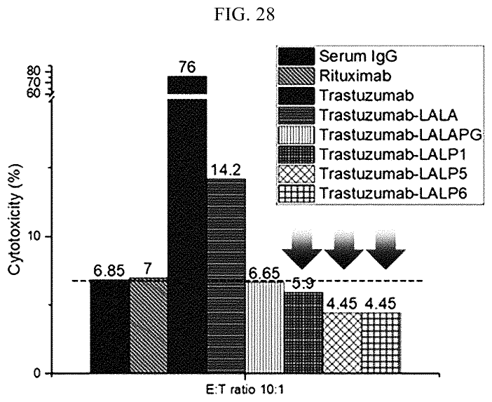

As a result, the glycosylated rituximab Fc variants of the present invention showed significantly lower cytotoxicity values than the conventional Fc variant LALA (L234A/L235A), thereby indicating that ADCC activity was significantly reduced. It was confirmed that the cytotoxicity value was lower than that of the LALAPG (L234A/L235A/P329G) Fc variants or human serum IgG used as a negative control (FIG. 28), thereby indicating effective removal of ADCC (FIG. 28).

### Example 22. Confirmation of ADCP Reduction of Glycosylated Trastuzumab Fc Variants

To confirm the reduced effector function of the glycosylated Fc variants of the present invention, antibody-dependent cell-mediated phagocytosis (ADCP) was analyzed. PBMCs were isolated from whole blood obtained from a healthy donor using Histopaque 1077, and monocytes were isolated by MACS (Miltenyi, 130-118-906) CD14 positive sorting. The isolated monocytes were treated with 50 ng/ml GM-CSF (PeproTech, 300-03) in RPMI medium containing 15% FBS and differentiated into macrophages for one week at 37° C. and 5% CO₂. SKBR-3 labeled with 2 μM PKH67 (Sigma Aldrich, MIDI67-1KT) was placed in a V-bottom plate at 2×10⁴ cells/well in RPMI medium diluted with 10% human serum, while differentiated macrophages were placed in the same plate at 1×10⁵ cells/well after recovering through Accutase (Innovative Cell Technologies, AT104), followed by incubation with antibody in each well for 4 hours at 37° C. and 5% CO₂. Afterwards, it was recovered through Accutase, washed with RPMI medium diluted with 10% human serum (Sigma Alrich, H6914-20ML), followed by fixation with 1% formaldehyde in a dark environment at room temperature for 10 minutes. Then, the cells were washed with 1×PBS and labeled with APC-anti-human CD14 antibody (BioLegend, 301807) and APC-CD11b (BioLegend, 301309) in a dark environment and on ice for 30 minutes. After washing, the fluorescence value was measured using BD FACS Lyric equipment, and phagocytosis (%) was expressed using the equation below.

$$Phagocytosis ( \% ) = Double positive / ( Target cell + Double positive ) \times 100$$

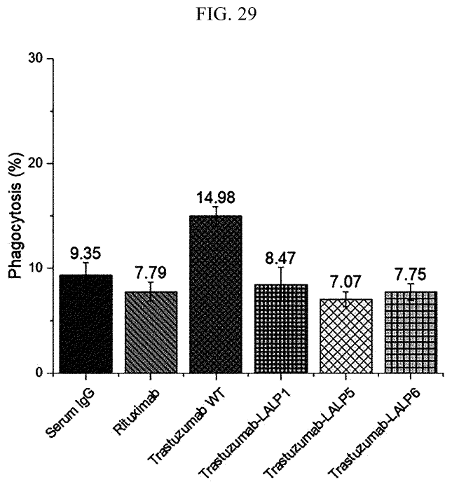

As a result, the glycosylated trastuzumab Fc variants of the present invention showed a lower phagocytosis rate than the wild-type trastuzumab Fc, thereby confirming that ADCP was removed (FIG. 29).

### Example 23. Confirmation of CDC Reduction of Glycosylated Rituximab Fc Variants

To confirm the reduced effector function of the glycosylated Fc variants of the present invention, complement-dependent cytotoxicity (CDC) was analyzed using Daudi, a cancer cell line expressing the target antigen of rituximab. In a V-bottom 96-well plate, 5×10⁵ cell/well of Daudi and each antibody were input into no phenol red RPMI-1640 (Gibco, 11835030) medium containing 20% human complement serum (Sigma-Aldrich, S1764), and then incubated for 1 hour at 37° C. under 5% CO₂. After incubation, lysed cells were labeled for 20 minutes at room temperature with FITC Annexin V Apoptosis Detection Kit with 7-AAD (Biolegend, 640922) prepared in Annexin V binding buffer (Biolegend, 422201) in a dark environment. Afterwards, it was washed with Annexin V binding buffer, fixed with 3% formaldehyde at room temperature for 10 minutes, and then washed in the same manner. The diluted samples were measured for fluorescence value using BD FACS Lyric equipment and analyzed for cytotoxicity (%) using the equation below.

Cytotoxicity (%)=(Pro-apoptotic cell+Dead cell)/(Total cell)×100−Background

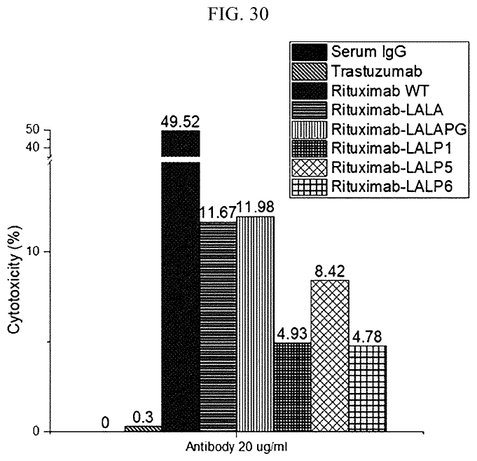

As a result, the glycosylated rituximab Fc variants of the present invention showed significantly lower cytotoxicity values than the conventional Fc variant LALA (L234A/L235A) and LALAPG (L234A/L235A/P329G) Fc variants. Further, it was confirmed that there was an excellent effect in reducing CDC (FIG. 30).

### Example 24. Activity Analysis of Glycosylated Trastuzumab Fc Variants in Mice

To confirm FcRn binding ability of the glycosylated trastuzumab Fc variants of the present invention, which is associated with the in vivo half-life in mice, ELISA assay was performed using mouse FcRn-GST by the method of Example 16 above.

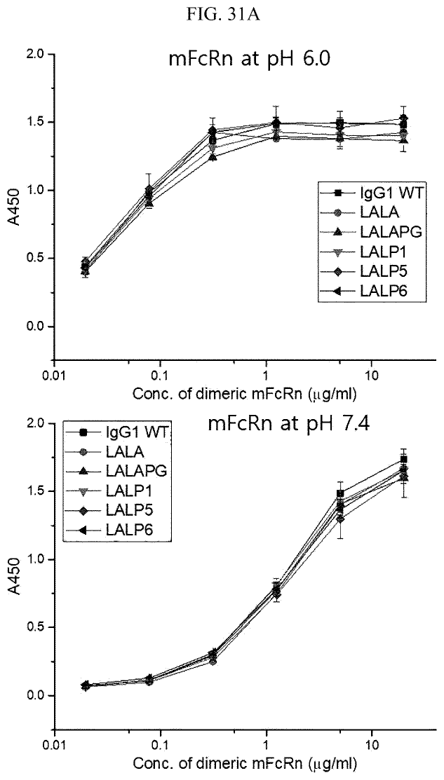

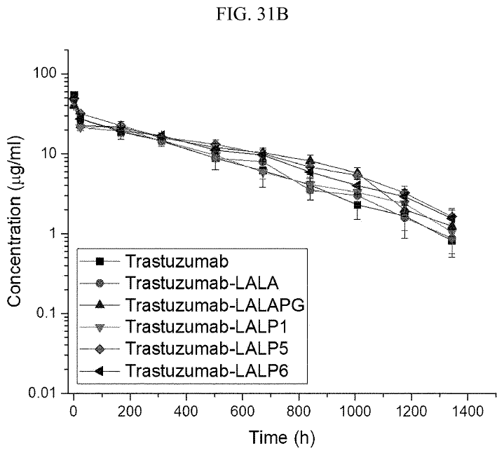

As a result, the trastuzumab Fc variants of the present invention were shown to maintain the FcRn binding properties of wild-type IgG1 (FIG. 31A).

Further, in vivo PK was analyzed to understand the interaction of the glycosylated trastuzumab Fc variants of the present invention in an in vivo system. An antibody was intravenously injected into C57BL/6 mice at 2 mg/kg using 5 mice per variant. 1, 24, 168, 312, 504, 672, 840, 1,008, 1,176 and 1,344 hours after injection, blood was collected from each mouse followed by centrifugation at 1,000×g for 15 minutes thus to ensure a serum from the supernatant. The serum was stored at −80° C. and then analyzed through ELISA to measure an antibody concentration in serum. After coating a high binding plated with HER2 diluted with 4 μg/ml in 0.05 M Na₂CO₃ (pH 9.6), it was fixed 4° C. for 16 hours. After blocking with 100 μl of 4% skim milk (GenomicBase, SK1400) at room temperature for 1 hour and washing 4 times with 180 μl of 0.05% PBST, standards and serum serially diluted with 1% skim milk were dispensed into each well and reacted at room temperature for 1 hour. After washing, 50 μl of goat human IgG H+L (Jackson Immunoresearch, 109-036-003) was added, followed by incubation at room temperature for 1 hour, and then washing. Subsequently, after color development by adding 50 μl of 1-Step Ultra TMB-ELISA substrate solution (Thermo Fisher Scientific, 34028), 50 μl of 2 M H₂SO₄ was added at a time to terminate the reaction, and then the absorbance at 450 nm was measured using Epoch microplate spectrophotometer (BioTek) equipment. The antibody concentration was analyzed from a standard curve to determine each concentration of trastuzumab and the glycosylated trastuzumab Fc variants over time. As a result, it was confirmed that the glycosylated trastuzumab Fc variants of the present invention may maintain a concentration similar to wild-type trastuzumab and have in vivo half-life (FIG. 31B).

### Example 25. Analysis of Mouse FcγRs Binding Ability of Glycosylated Trastuzumab Fc Variants

The use of the glycosylated trastuzumab Fc variant of the present invention was observed in animal experiments. ELISA assay was performed to confirm the binding ability of the glycosylated trastuzumab Fc variant to mouse FcγRs using mouse FcγRs-GST according to the method in Example 11 above.

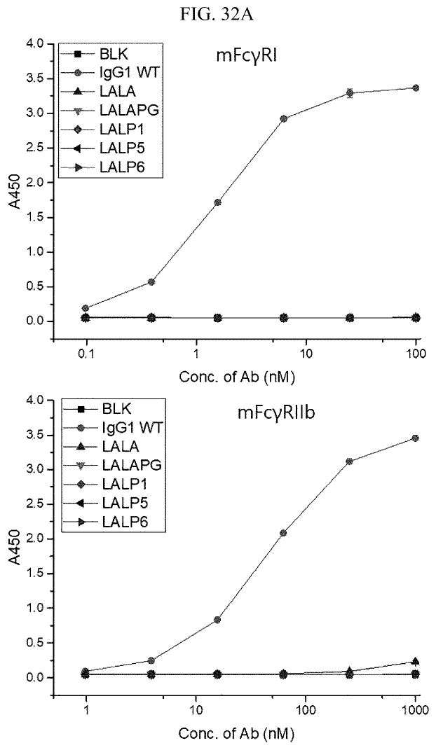

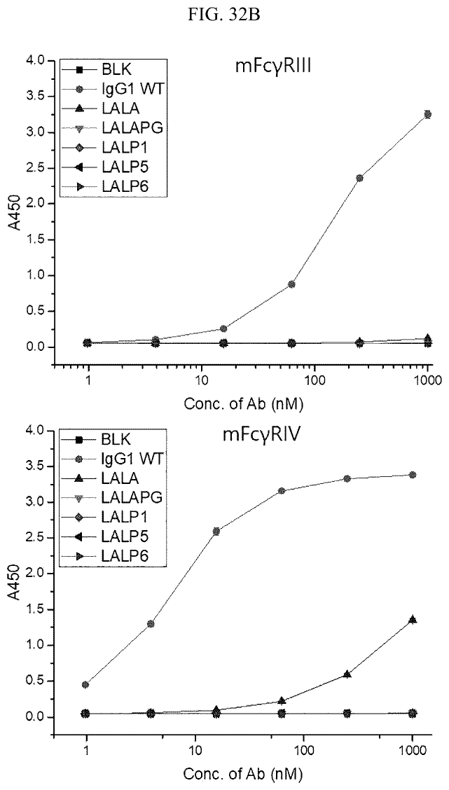

As a result, it was confirmed that the binding ability of each of the glycosylated trastuzumab Fc variants (LALP1, LALP5 and LALP6) of the present invention to mFcγRI, mFcγRIIb and mFcγRIII was completely removed. Further, even with regard to mFcγRIV, to which the LALA mutant (L234A/L235A) has strong binding ability, the inventive variants showed the binding ability completely removed (FIGS. 32A and 32B).

### Example 26. Analysis of Cynomolgus Monkey FcγRs Binding Ability of Glycosylated Trastuzumab Fc Variants

The use of the glycosylated trastuzumab Fc variant of the present invention was observed in animal experiments. ELISA assay was performed to confirm the binding ability of the glycosylated trastuzumab Fc variant to cynomolgus monkey FcγRs using the cynomolgus monkey FcγRs-GST according to the method in Example 11 above.

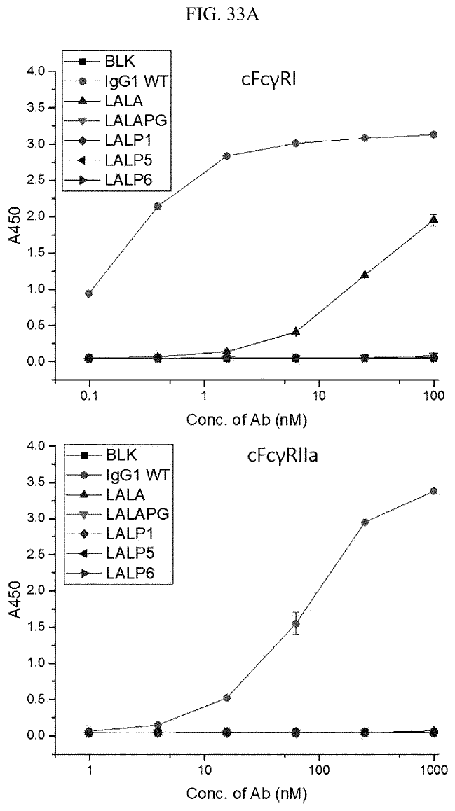

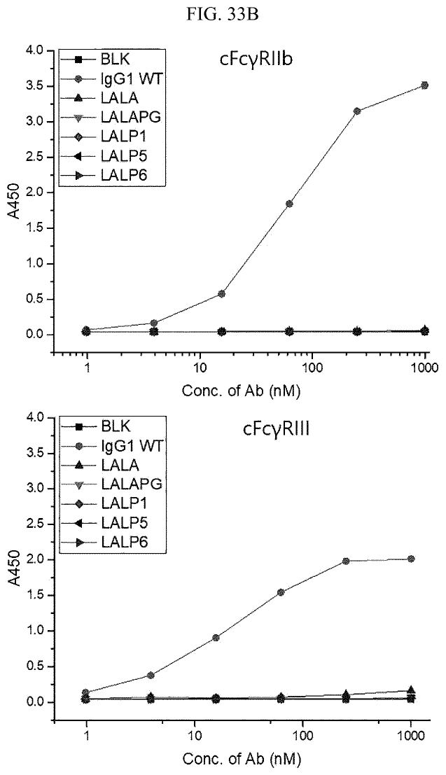

As a result, it was confirmed that the binding ability of the glycosylated trastuzumab Fc variants (LALP1, LALP5 and LALP6) of the present invention to cFcγRIIa, cFcγRIIb and cFcγRIII was completely removed. Further, even with regard to cFcγRI and cFcγRIII, to which the LALA mutant (L234A/L235A) has strong binding ability, the inventive variants showed the binding ability completely removed (FIGS. 33A and 33B).

Binding affinities to human FcγRs, mouse FcγRs, cynomolgus monkey FcγRs and human C1q, and FcRn; thermal stability (Tm), ADCC activity and CDC activity were compared between the glycosylated trastuzumab Fc variants (LALP1, LALP5 and LALAP6) of the present invention and the IgG1 wild-type, LALA variant and LALAPG variant (Tables 6 and 7).

TABLE 6

| | | | hFcγRIIa | hFcγRIIa | | hFcγRIIIa | hFcγRIIIa | | | Tm | | | |
|---|---|---|---|---|---|---|---|---|---|---|---|---|---|
| | | hFcγRI | (H) | (R) | hFcγRIIb | (V) | (F) | hC1q | FcRn | (° C.) | ADCC | ADCP | CDC |
| Control | IgG1 WT | ++++ | +++ | +++ | ++ | +++ | +++ | +++ | +++ | 69.54 | +++ | ++ | +++ |
| Control | LALA | ++ | + | + | + | ++ | ++ | − | +++ | 69.83 | ++ | − | + |
| Control | LALALPG | + | − | − | − | − | + | + | +++ | 67.35 | + | − | + |
| Variant | LALP1 | − | − | − | − | − | − | − | +++ | 67.04 | − | − | − |
| Variant | LALP5 | − | − | − | − | − | − | − | +++ | 68.93 | − | − | − |
| Variant | LALP6 | − | − | − | − | − | − | − | +++ | 68.50 | − | − | − |

TABLE 7

| | | mFcγRI | mFcγRIIb | mFcγRIII | mFcγRIV | cFcγRI | cFcγRIIa | cFcγRIIb | cFcγRIII |
|---|---|---|---|---|---|---|---|---|---|
| Control | IgG1 WT | +++ | ++ | ++ | +++ | +++ | ++ | ++ | ++ |
| Control | LALA | − | + | − | ++ | ++ | − | − | + |
| Control | LALALPG | − | − | − | − | − | − | − | − |
| Variant | LALP1 | − | − | − | − | − | − | − | − |
| Variant | LALP5 | − | − | − | − | − | − | − | − |
| Variant | LALP6 | − | − | − | − | − | − | − | − |

1: A human antibody Fc domain variant in which amino acid 235 of the human antibody Fc domain is replaced with proline (P).

2: The human antibody Fc domain variant according to claim 1, wherein the human antibody Fc domain comprising an amino acid sequence of SEQ ID NO: 15.

3: The human antibody Fc domain variant according to claim 1, wherein amino acid 234 of the human antibody Fc domain is further substituted with alanine (A).

4: The human antibody Fc domain variant according to claim 1, wherein amino acid 265 of the human antibody Fc domain is further substituted with leucine (L), methionine (M) or asparagine (N).

5: The human antibody Fc domain variant according to claim 1, wherein amino acid 329 of the human antibody Fc domain is further substituted with valine (V), leucine (L), or glycine (G).

6: The human antibody Fc domain variant according to claim 1, wherein amino acid 268 of the human antibody Fc domain is further substituted with glutamine (Q).

7: The human antibody Fc domain variant according to claim 1, wherein amino acid 296 of the human antibody Fc domain is further substituted with phenylalanine (F).

8: The human antibody Fc domain variant according to claim 1, wherein amino acid 300 of the human antibody Fc domain is further substituted with phenylalanine (F).

9: The human antibody Fc domain variant according to claim 1, wherein amino acid 333 of the human antibody Fc domain is further substituted with valine (V).

10: The human antibody Fc domain variant according to claim 1, wherein the human antibody is IgG1.

11: An antibody comprising the human antibody Fc domain variant according to claim 1, or a fragment thereof having immunological activity.

12: An antibody-drug conjugate in which the antibody according to claim 11 or a fragment thereof having immunological activity is conjugated to one or more drugs.

13: The antibody-drug conjugate according to claim 12, wherein the drug is any one selected from the group consisting of: chimeric antigen receptor (CAR) cell therapeutics, oncolytic drug, immunotherapy agent, cytotoxic agent, angiogenesis inhibitor, kinase inhibitor, costimulatory molecule blocker, adhesion molecule blocker, anti-cytokine agent, nucleic acid therapeutics, anti-CTLA-4 agent, anti-PD-1 agent, anti-PD-L1 agent, anti-PD-L2 agent, TNF-α cross-linking agent, TRAIL cross-linking agent, anti-CD27 agent, anti-CD30 agent, anti-CD40 agent, anti-4-1BB agent, anti-GITR agent, anti-OX40 agent, anti-TRAILR1 agent, anti-TRAILR2 agent, tagretin, interferon-alpha, clobetasol, peg interferon, prednisone, romidepsin, bexarotene, methotrexate, triamcinolone cream, anti-chemokine, vorinostat, gabapentin, cyclosporine, rapamycin, FK506, detectable marker or reporter, TNF antagonist, antirheumatic agent, muscle relaxants, narcotics, non-steroidal anti-inflammatory drugs (NSAIDs), analgesics, anesthetics, sedatives, local anesthetics, neuromuscular blocker, antibacterial agent, psoriasis therapeutics, corticosteroid, anabolic steroid, erythropoietin, immunizations, immunoglobulin, immunosuppressant, growth hormone, hormone replacement drug, radiopharmaceuticals, antidepressant, antipsychotics, stimulant, asthma drug, beta agonist, inhaled steroid, epinephrine or its analogues, cytokine, cytokine antagonist, PD-1 antagonist, adenosine A2AR antagonist, CD73 inhibitor, CTLA-4 inhibitor, TIM-3 inhibitor, LAG-3 inhibitor, anthracycline, antisense oligonucleotide (ASO), siRNA and aptamer.

14: A pharmaceutical composition comprising:
the human antibody Fc domain variant according to claim 1;
an antibody including the same or a fragment thereof having immunological activity; or
an antibody-drug conjugate comprising a drug and the antibody conjugated to the drug.

15: The pharmaceutical composition according to claim 14, wherein the cancer is selected from the group consisting of: brain tumor, melanoma, myeloma, non-small cell lung cancer, oral cancer, liver cancer, stomach cancer, colon cancer, breast cancer, lung cancer, bone cancer, pancreatic cancer, skin cancer, head or neck cancer, cervical cancer, ovarian cancer, colon cancer, small intestine cancer, rectal cancer, fallopian tube carcinoma, anal cancer, endometrial carcinoma, vaginal carcinoma, vulvar carcinoma, Hodgkin's disease, esophageal cancer, lymph node cancer, bladder cancer, gallbladder cancer, endocrine cancer, thyroid cancer, parathyroid cancer, adrenal cancer, soft tissue sarcoma, urethral cancer, penile cancer, prostate cancer, chronic or acute leukemia, lymphocytic lymphoma, renal or ureteral cancer, renal cell carcinoma, renal pelvic carcinoma, central nervous system tumor, primary central nervous system lymphoma, spinal cord tumor, brain stem glioma and pituitary adenoma.

16: A method for treatment of cancer, comprising:
administering the human antibody Fc domain variant according to claim 1, an antibody including the same or a fragment thereof having immunological activity; and/or an antibody-drug conjugate comprising a drug and the antibody conjugated to the drug, to a subject.

17: The method according to claim 16, wherein the cancer is selected from the group consisting of: brain tumor, melanoma, myeloma, non-small cell lung cancer, oral cancer, liver cancer, stomach cancer, colon cancer, breast cancer, lung cancer, bone cancer, pancreatic cancer, skin cancer, head or neck cancer, cervical cancer, ovarian cancer, colon cancer, small intestine cancer, rectal cancer, fallopian tube carcinoma, anal cancer, endometrial carcinoma, vaginal carcinoma, vulvar carcinoma, Hodgkin's disease, esophageal cancer, lymph node cancer, bladder cancer, gallbladder cancer, endocrine cancer, thyroid cancer, parathyroid cancer, adrenal cancer, soft tissue sarcoma, urethral cancer, penile cancer, prostate cancer, chronic or acute leukemia, lymphocytic lymphoma, renal or ureteral cancer, renal cell carcinoma, renal pelvic carcinoma, central nervous system tumor, primary central nervous system lymphoma, spinal cord tumor, brain stem glioma and pituitary adenoma.

18: A composition for target detection, comprising a conjugate in which the antibody according to claim 11 or a fragment thereof having immunological activity and a functional substance are bound.

19: The composition for target detection according to claim 18, wherein the functional material is one or more selected from the group consisting of contrast agents, radioisotopes, inorganic particles, fluorescent markers, dyes, enzymes, polypeptides, nucleic acids, carbohydrates and lipids.

20: A method for diagnosing a disease, comprising administering the composition for target detection according to claim 18 to an individual.

21: The method according to claim 20, wherein the disease is any one selected from the group consisting of cancer, cardiovascular disease, neurological disorder, diabetes, autoimmune disease, inflammatory disease, viral infectious disease and allergy.


SEQUENCE LISTING (21 sequences)

SEQ ID NO: 1 - protein, 227 aa, synthetic construct
DKTHTCPPCPAPEAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - DNA, 681 bp, synthetic construct
  source 1..681: mol_type = other DNA
gacaaaactcacacatgcccaccgtgcccagcacctgaagctcccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggacgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 3 - protein, 227 aa, synthetic construct
DKTHTCPPCPAPEAPGGPSVFLFPPKPKDTLMISRTPEVTCVVVNVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - DNA, 681 bp, synthetic construct
  source 1..681: mol_type = other DNA
gacaaaactcacacatgcccaccgtgcccagcacctgaagctcccgggggaccgtcagtcttcctcttccccccaaaacccaaggacaccctcatgatctcccggacccctgaggtcacatgcgtggtggtgaatgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggacgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 5 - protein, 227 aa, synthetic construct
DKTHTCPPCPAPEAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALVAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - DNA, 681 bp, synthetic construct
  source 1..681: mol_type = other DNA
gacaaaactcacacatgcccaccgtgcccagcacctgaagctcccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcgtggcccccatcgagaaaaccatctccaaagccaaagggcagccccgagaaccacaggtgtacaccctgcccccatcccgggacgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 7 - protein, 227 aa, synthetic construct
DKTHTCPPCPAPEAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALLAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - DNA, 681 bp, synthetic construct
  source 1..681: mol_type = other DNA
gacaaaactcacacatgcccaccgtgcccagcacctgaagctcccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcttggcccccatcgagaaaaccatctccaaagccaaagggcagccccgagaaccacaggtgtacaccctgcccccatcccgggacgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 9 - protein, 227 aa, synthetic construct
DKTHTCPPCPAPEAPGGPSVFLFPPKPKDTLMISRTPEVTCVVVMVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - DNA, 681 bp, synthetic construct
  source 1..681: mol_type = other DNA
gacaaaactcacacatgcccaccgtgcccagcacctgaagctcccgggggaccgtcagtcttcctcttccccccaaaacccaaggacaccctcatgatctcccggacccctgaggtcacatgcgtggtggtgatggtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggacgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 11 - protein, 227 aa, synthetic construct
DKTHTCPPCPAPEAPGGPSVFLFPPKPKDTLMISRTPEVTCVVVLVSHEDPEVKFNWYVDGVEVHNAKTKPREEQYNSTYRVVSVLTVLHQDWLNGKEYKCKVSNKALPALIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - DNA, 681 bp, synthetic construct
  source 1..681: mol_type = other DNA
gacaaaactcacacatgcccaccgtgcccagcacctgaagctcccgggggaccgtcagtcttcctcttccccccaaaacccaaggacaccctcatgatctcccggacccctgaggtcacatgcgtggtggtgcttgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagccttgatcgagaaaaccatctccaaagccaaagggcagccccgagaaccacaggtgtacaccctgcccccatcccgggacgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 13 - protein, 227 aa, synthetic construct
DKTHTCPPCPAPEAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - DNA, 681 bp, synthetic construct
  source 1..681: mol_type = other DNA
gacaaaactcacacatgcccaccgtgcccagcacctgaagctcccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccatcccgggacgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 15 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - DNA, 681 bp, Homo sapiens
  source 1..681: mol_type = genomic DNA
gacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggacgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 17 - protein, 76 aa, Capra hircus
PCKHRCPPITGKGQDQSFNTRFFAPIQTGFHEGVRRTAVAPQESSTLVTDYQRSEDYGTSQAYRRNSEDTYAVIKP

SEQ ID NO: 18 - protein, 7 aa, synthetic construct
ELPGGPS

SEQ ID NO: 19 - protein, 6 aa, synthetic construct
VDVSHE

SEQ ID NO: 20 - protein, 5 aa, synthetic construct
YNSTY

SEQ ID NO: 21 - protein, 6 aa, synthetic construct
LPAPIE